SEQUENCE LISTING (47 sequences)

SEQ ID NO: 1 - protein, 9 aa, Arabidopsis thaliana
TAVPIPLVG

SEQ ID NO: 2 - protein, 9 aa, Arabidopsis thaliana
AAVPIPLVG

SEQ ID NO: 3 - protein, 10 aa, Brassica rapa
YADVPIPLVG

SEQ ID NO: 4 - protein, 10 aa, Brassica rapa
YSTVPIPLVG

SEQ ID NO: 5 - protein, 10 aa, Brassica rapa var. glabra
SANVPIPLVG

SEQ ID NO: 6 - protein, 9 aa, Brassica oleracea var. capitata
SAIPVPLVG

SEQ ID NO: 7 - protein, 9 aa, Gossypium hirsutum
AAPPVPFVG

SEQ ID NO: 8 - protein, 63 aa, Arabidopsis thaliana
MVTLKMEICIELLKLTVDFVAAVAESIEVAFRHRPPPVIQYSSVMYGRRSNYTAVPIPLVGFL

SEQ ID NO: 9 - protein, 60 aa, Arabidopsis thaliana
MVSQKLEICIELVKIAVVFVTTVAESVEEAFRKPPPSLPAVHGGRRNNYAAVPIPLVGFM

SEQ ID NO: 10 - protein, 60 aa, Thlaspi caerulescens
  misc_feature (27)..(28): Xaa can be any naturally occurring amino acid
MVSQKLEICIELMKITVLFVTTVAESXXEVFCKXPPAHPAVHGGRRNXXAAVPIPLXGFM

SEQ ID NO: 11 - protein, 60 aa, Thlaspi caerulescens
MVSQKLEICIKLMKITVLFVTTVAESVEEVFCKSPPALPAVHGGRRNSYAAVPIPLVGFM

SEQ ID NO: 12 - protein, 64 aa, Thellungiella halophila
MVNLKLEICIEFVKLTVDFVAAVAESIEVAFRHRPPPTIPYSAVMNGRRSNYSAAVPIPLVGFL

SEQ ID NO: 13 - protein, 60 aa, Brassica rapa
MVSQKLEICIELVKIGVVFVATVAESVEKAFRKPPPSLPAVHDSRRNNYADVPIPLVGFM

SEQ ID NO: 14 - protein, 59 aa, Brassica rapa
MVSQKLEICIELMKIGVVFVATVAESLKAFRKPPPSLPAVHDSRRNSYADVPIPLVGFM

SEQ ID NO: 15 - protein, 63 aa, Brassica rapa
MVTLKLEICIEFVKLTVDLVAAMAESIEVAFRHRPPPQVPYSAGVNGRRGHYSTVPIPLVGFL

SEQ ID NO: 16 - protein, 60 aa, Brassica rapa var. glabra
MVSQKLEFCIELVKMAVVFVAAVAESLEEAFRKPQPALPVVHDGRRNSYANVPIPLVGFM

SEQ ID NO: 17 - protein, 63 aa, Brassica oleracea var. capitata
MVTMKLEICIELVKLTVDFVAAVAESIEVAFRNRPPPPIQHLAVGNGRRSNHSAIPVPLVGFL

SEQ ID NO: 18 - protein, 65 aa, Gossypium hirsutum
MVLQRLEICVQMVKLVIEFVIVVAEAVGIVIHQNDSNRPVIMSMPSRSFAAPPVPFVGFLPYSGF

SEQ ID NO: 19 - DNA, 192 bp, Arabidopsis thaliana
atggtgactctaaaaatggagatttgcatcgagttgttgaaattgaccgtagatttcgtcgccgccgttgcagaatcaatcgaagtagctttccgccatcgtcctccaccggtgattcaatattcttcagtgatgtatggtcgccggagtaattacaccgccgttcccattcctctcgtggggtttctgtag

SEQ ID NO: 20 - DNA, 183 bp, Arabidopsis thaliana
atggtgtctcaaaagctagagatttgcatcgaattggtgaaaattgctgttgttttcgtcaccaccgtggctgaatcagtagaagaagctttccgtaagcctccaccgtctcttccagcagttcacggtggtcgccggaataattacgccgccgttcccattcctcttgttggattcatgtga

SEQ ID NO: 21 - DNA, 183 bp, Thlaspi caerulescens
  misc_feature (79)..(79): n is a, c, g, or t
atggtatctcaaaagctggagatttgcatcgaattgatgaagattaccgtccttttcgtcaccaccgtggctgaatcantanaagaagttttttgtaagtntccaccggcgcatccagcagttcacggtggtcgccggaatanttncgccgccgttcccattccgcttgntggatttatgtga

SEQ ID NO: 22 - DNA, 183 bp, Thlaspi caerulescens
atggtgtctcaaaagctggagatttgcatcaaattgatgaagattaccgtccttttcgtcaccaccgtggctgaatcagtagaagaagttttttgtaagtctccaccggcgcttccagcagttcacggtggtcgccggaatagttacgccgccgttcccattccgcttgttggatttatgtga

SEQ ID NO: 23 - DNA, 195 bp, Thellungiella halophila
atggtgaatttaaaacttgagatttgcatcgagtttgtgaaattaaccgtagatttcgtcgccgccgtagcggaatcgatcgaagtagctttccgacaccgtcctccgccaactattccgtattcagctgtgatgaacggtcgccggagtaattactccgccgccgttcccattcctctcgtggggtttctgtga

SEQ ID NO: 24 - DNA, 183 bp, Brassica rapa
atggtgtctcaaaagctagagatctgcatcgaattagtgaagattggcgtcgtttttgtcgccactgtggctgaatcagtagaaaaagctttccgtaagccaccaccgtcgcttccggcagttcatgatagtcgccggaacaattacgccgacgttcctattcctctcgttggattcatgtga

SEQ ID NO: 25 - DNA, 183 bp, Brassica rapa
atggtgtctcaaaagctagagatctgcatcgaattaatgaagattggcgtcgtttttgtcgccactgtggctgaatcattataaaaagctttccgtaagccaccaccgtcgcttccggcagttcatgatagtcgccggaacagttacgccgacgttcctattcctcttgttggattcatgtga

SEQ ID NO: 26 - DNA, 192 bp, Brassica rapa
atggtgactctaaagcttgagatttgcatcgagtttgtgaaactaaccgttgatctcgtcgccgccatggcagaatcaatcgaagtagctttccgtcaccgtcctccgccgcaggttccatattcagcgggggtgaacggtcggcggggtcattactccaccgttcccattcctctcgtaggctttctgtaa

SEQ ID NO: 27 - DNA, 183 bp, Brassica rapa var. glabra
atggtgtctcaaaagctggagttttgcatcgaattagtgaagatggcagtcgtttttgtcgccgccgtggctgaatcattagaagaagctttccgtaagcctcagccggcgcttccggtagttcatgacggtcgccggaacagttacgctaacgttcccattcctctcgttggattcatgtga

SEQ ID NO: 28 - DNA, 192 bp, Brassica oleracea var. capitata
atggtgactatgaaacttgagatttgcatagagttggtgaaactaacagtagatttcgtcgctgccgtggcagaatcaatcgaagtagctttccgtaaccgtcctccgccgcctattcaacatttagcggtggggaacggccgtcggagtaatcactccgccatccccgttcctctagtgggatttctgtaa

SEQ ID NO: 29 - DNA, 195 bp, Gossypium hirsutum
atggtgctgcagagattagagatatgcgtgcagatggtgaaactagtgatagagtttgtcatcgttgtggctgaagctgttgggattgtcattcaccaaaatgatagtaaccggcccgtcataatgtcgatgcctagccgctcttttgctgctcctccggttccatttgttgggtttcttccttattctgggttt

SEQ ID NO: 30 - protein, 14 aa, Arabidopsis thaliana
TASYPSPFLFGFLP

SEQ ID NO: 31 - protein, 15 aa, Brassica rapa
ASTPRPSPYMIGYLP

SEQ ID NO: 32 - protein, 15 aa, Thellungiella halophila
ASFSHPSPYIIGYLP

SEQ ID NO: 33 - protein, 17 aa, Arabidopsis thaliana
SASPNPPSSHYIIGFLP

SEQ ID NO: 34 - protein, 67 aa, Arabidopsis thaliana
MVIKRIELCIELMKIGMEFVVVVAEAVQIVMRQHLNHRAPPPPPVLRQGLLPYTASYPSPFLFGFLP

SEQ ID NO: 35 - protein, 65 aa, Brassica rapa
MLIKRIELCIEIVKKTMDFAVVVAEAAKVFLRNAPQAPPALLRQGPYYYSASTPRPSPYMIGYLP

SEQ ID NO: 36 - protein, 65 aa, Thlaspi caerulescens
MVIKRIEMCIEMVKIAKELVVVVSEAVRVFLSNAPPTPPALLRHGPYHYSASFSHPSPYIIGYLP

SEQ ID NO: 37 - protein, 66 aa, Arabidopsis thaliana
MVIKKIELCIEITKITMEFLVVVADAVTIFLRQSSPPPPALLRHGLYSYSASPNPPSSHYIIGFLP

SEQ ID NO: 38 - DNA, 204 bp, Arabidopsis thaliana
atggtgataaaaaggatagagctgtgtatagagctaatgaagataggtatggagtttgtagtggtggtggctgaagccgtccagattgttatgcggcaacacctcaaccaccgtgctccacctcctccgcctgtcctccgccagggtcttctcccttataccgcttcttacccttctccattcctatttggatttctcccttga

SEQ ID NO: 39 - DNA, 198 bp, Brassica rapa
atgttgataaaaagaatagagctctgtatagagatagtgaagaaaacaatggattttgcggtagtggtggctgaggccgcgaaagtcttcttgaggaacgctcctcaggctccgcccgctctcctacgccaaggcccctactattactccgcctcgacccctcgtccttccccttacatgattggctatcttccatga

SEQ ID NO: 40 - DNA, 198 bp, Thellungiella halophila
atggtgataaagagaatagagatgtgtatagagatggtgaagatagcgaaggagttagtggtggtggtgtctgaagccgtgagagtcttcttgagcaacgctcctccgactcctccggccctcctccgccacggcccctaccattactccgcctccttctctcacccttccccttatatcatcggttatcttccctga

SEQ ID NO: 41 - DNA, 201 bp, Arabidopsis thaliana
atggtgataaagaaaatcgagctatgcatagagattacgaagataacaatggagttcttggtggtggtggctgacgccgtcacaatcttcttaaggcaatcttctccacctcctccggctctactccgccatggtctctactcatactctgcctcccctaatcctccttcttcccattacatcatcggctttcttccctaa

SEQ ID NO: 42 - DNA, 18 bp, Artificial
  primer
tagctttccgccatcgtc

SEQ ID NO: 43 - DNA, 20 bp, Artificial
  primer
tacagaaaccccacgagagg

SEQ ID NO: 44 - DNA, 26 bp, Artificial
  primer
caccatggtgactctaaaaatggaga

SEQ ID NO: 45 - DNA, 20 bp, Artificial
  primer
ctacagaaaccccacgagag

SEQ ID NO: 46 - DNA, 18 bp, Artificial
  primer
gagcggtaggcgtaggtc

SEQ ID NO: 47 - DNA, 20 bp, Artificial
  primer
gcccaccagagtgtatgagt

(19) United States
(12) Patent Application Publication
(10) Pub. No.: US 2014/0143910 A1
(43) Pub. Date: May 22, 2014

(54) COMPOSITION THAT AUGMENTS PLANT DISEASE RESISTANCE AND/OR BRANCHING

(71) Applicants: Takeshi Nakano, Saitama (JP); Tadao Asami, Saitama (JP); Ayumi Yamagami, Saitama (JP)

(72) Inventors: Takeshi Nakano, Saitama (JP); Tadao Asami, Saitama (JP); Ayumi Yamagami, Saitama (JP)

(73) Assignee: Riken, Saitama (JP)

(21) Appl. No.: 13/983,960

(22) PCT Filed: Feb. 7, 2012
(86) PCT No.: PCT/JP2012/052720

§ 371 Date: Oct. 15, 2013

(30) Foreign Application Priority Data
Feb. 7, 2011 (JP) 2011-024394

(51) Int. Cl. A01N 43/36; C12N 15/82

(52) U.S. Cl. CPC A01N 43/36; C12N 15/8279
USPC 800/301; 530/328; 530/324; 504/287

(57) ABSTRACT

The objective of the present invention is to develop and provide: a composition that more inexpensively and safely augments plant disease resistance and/or plant branching by inducing new plant disease resistance via a brassinosteroids; and a method for suppressing microbial infection of plants and a method for augmenting branching using the composition. Provided is a composition containing as the active ingredient a peptide hormone obtained on the basis of isolating a disease resistant brassinosteroid variant and analyzing the causative gene thereof.

### TECHNICAL FIELD

The present invention relates to a composition that augments plant disease resistance to microbial infection, etc., and/or plant branching, and a method for suppressing infectious disease in a plant and a method for augmenting plant branching using the same.

### BACKGROUND ART

Microbial infection is inevitable for plants and usually causes serious stress to plant bodies. Against such microbial infection, particularly, pathogenic infection, plants have evolved their own protective or defensive systems, in addition to morphological adaptation. Specifically, the primary response of each plant to the pathogenic infection involves the specific recognition of the pathogens and the rapid induction of cell death (hypersensitive cell death) of infected cells to eliminate the pathogens together with the infected cells (Non Patent Literature 1). The secondary response of the plant is the induction of pathogen resistance called systemic acquired resistance (SAR), which is triggered by the hypersensitive cell death, in order to protect the plant body from further attacks by the pathogens (Non Patent Literatures 2 and 3). SAR has been confirmed in many plants and confers resistance to various plant pathogens to the uninfected portions of plants (Non Patent Literatures 4 and 5). Salicylic acid has been identified as a signaling factor inducing this SAR, in dicot plants such as Arabidopsis thaliana and tobacco (Non Patent Literatures 6 and 7). If other signaling factors inducing SAR are identified and the intracellular signals of plants can be controlled, disease resistance can be conferred to plant bodies without being mediated by hypersensitive cell death. Unfortunately, much remains to be revealed about this SAR-inducing signaling mechanism mediated by salicylic acid, and the whole picture of the mechanism has not yet been clarified.

### CITATION LIST

### Non Patent Literature

Non Patent Literature 1: Ross, A. F. (1961) Virology, 14: 340-358
Non Patent Literature 2: Kuc J. (1982) Bioscience, 32: 854-860
Non Patent Literature 3: McIntyre J. L. et al., (1981) Phytopathology, 71: 297-301
Non Patent Literature 4: Chester K S., (1933) Q. Rev. Biol., 8: 275-324
Non Patent Literature 5: Durner J. et al., (1997) Trends in Plant Sci., 2: 266-274
Non Patent Literature 6: Delaney T P. et al., (1994) Science, 266: 1247-1250
Non Patent Literature 7: Gaffney T. et al., (1993) Science, 261: 754-756

### SUMMARY OF INVENTION

### Technical Problem

The present inventors have newly found that brassinosteroid can induce the disease resistance of a plant through a pathway different from the salicylic acid-mediated SAR-inducing signaling mechanism described above (Nakashita H. et al., 2003, The Plant Jour., 33: 887-898). Brassinosteroid (hereinafter, referred to as “BR”) is a phytohormone that is involved in the regulation of plant growth, photomorphogenesis, the control of vascular bundle formation, the functional regulation of chloroplasts, etc., and plays a principal role in various areas of plant growth cycles (Azpiroz R. et al., 1988, Plant Cell, 10: 219-230; Clouse S. & Sasse J., 1998, Annu. Rev. Plant Physiol. Plant Mol. Biol., 49: 427-451; Mandava N., 1988, Annu. Rev. Plant Physiol. Plant Mol. Biol., 39: 23-52; and Sakurai A. et al., 1999, Brassinosteroids, Steroidal Plant Hormones, Tokyo: Springer). On the basis of the novel BR-mediated disease resistance of a plant (brassinosteroid-mediated disease resistance; hereinafter, referred to as “BDR”), the development of a plant disease resistance-augmenting agent comprising BR as an active ingredient has been expected. BR, however, is synthesized by complicated steps and thus, is disadvantageously unpractical in terms of production cost in the agricultural field where such an agent is used in large amounts.

Thus, an object of the present invention is to develop and provide a novel plant disease resistance-augmenting agent that is capable of inducing BDR instead of BR and augmenting plant disease resistance in a more inexpensive way, and a method for preventing and treating infectious disease in a plant using the same.

### Solution to Problem

To attain the object, the present inventors have predicted that a signaling factor (hereinafter, referred to as a “BR intracellular signaling factor”) that functions in an intracellular signaling pathway activated by BR (hereinafter, referred to as a “BR intracellular signaling pathway”) may also be involved, as in BR, in the induction of BDR-induced disease resistance. On the basis of this hypothesis, the present inventors have isolated many mutants involved in the pathway using Arabidopsis thaliana. Results of particularly analyzing a bil3 mutant, one of brassinosteroid signaling pathway mutants bil (Brz-insensitive-long hypocotyl) having resistance to a BR biosynthesis inhibitor brassinazole (Brz), have demonstrated that the BIL3 gene encodes a novel peptide hormone capable of being extracellularly secreted and its overexpression not only induces BDR but also increases the number of branching in the plant body. The control of plant branching is important for, for example, the control of the yields of agricultural or horticultural crops. The present invention is based on these new findings and provides the followings:

(1) A peptide consisting of a following amino acid sequence and having activities of augmenting plant disease resistance and/or branching, or a salt thereof:

(a) the amino acid sequence represented by SEQ ID NO: 1, or

(b) an amino acid sequence having identity to 4 or more amino acids in the amino acid sequence represented by SEQ ID NO: 1 and having

an alanine residue or a serine residue as an amino acid residue corresponding to position 2 of the amino acid sequence represented by SEQ ID NO: 1, nonpolar amino acid residues as amino acid residues corresponding to positions 3, 5, 7, and 8 thereof, proline residues as amino acid residues corresponding to positions 4 and 6 thereof, and a glycine residue as an amino acid residue corresponding to position 9 thereof.

(2) The peptide or the salt thereof according to (1), wherein the amino acid sequence (b) further has a valine residue, an isoleucine residue, or a proline residue as an amino acid residue corresponding to position 3 of the amino acid sequence represented by SEQ ID NO: 1, an isoleucine residue or a valine residue as an amino acid residue corresponding to position 5 thereof, a leucine residue or a phenylalanine residue as an amino acid residue corresponding to position 7 thereof, and/or a valine residue as an amino acid residue corresponding to position 8 thereof.

(3) The peptide or the salt thereof according to (1) or (2), wherein the amino acid sequence (b) has an alanine residue as an amino acid residue corresponding to position 2 of the amino acid sequence represented by SEQ ID NO: 1, a valine residue as an amino acid residue corresponding to position 3 thereof, an isoleucine residue as an amino acid residue corresponding to position 5 thereof, and/or a leucine residue as an amino acid residue corresponding to position 7 thereof.

(4) The peptide or the salt thereof according to (1), wherein the peptide having the amino acid sequence (b) consists of any of the amino acid sequences represented by SEQ ID NOs: 2 to 7.

(5) The peptide or the salt thereof according to (1), wherein the peptide having the amino acid sequence (b) consists of any of the amino acid sequences represented by SEQ ID NOs: 8 to 18.

(6) A composition for conferring disease resistance to a plant and/or for augmenting plant branching, comprising at least one of a peptide according to any of (1) to (5), a peptide according to any of (1) to (5) further having amino acid(s) added to the N terminus and/or the C terminus thereof, and salts thereof, as an active ingredient.

(7) A method for suppressing microbial infection in a plant, comprising the step of allowing a peptide or a salt thereof according to any of (1) to (5) and/or a composition according to (6) to act on the plant.

(8) A method for augmenting plant branching, comprising the step of allowing a peptide or a salt thereof according to any of (1) to (5) and/or a composition according to (6) to act on the plant.

(9) A plant with conferred disease resistance and/or augmented branching, comprising at least one exogenous nucleic acid expression system comprising, in an expressible state, a nucleic acid encoding a following peptide having activities of augmenting plant disease resistance and/or branching:

(a) a peptide that comprises the amino acid sequence represented by SEQ ID NO: 1; or

(b) a peptide that comprises an amino acid sequence having identity to 4 or more amino acids in the amino acid sequence represented by SEQ ID NO: 1 and having an alanine residue or a serine residue as an amino acid residue corresponding to position 2 of the amino acid sequence represented by SEQ ID NO: 1, nonpolar amino acid residues as amino acid residues corresponding to positions 3, 5, 7, and 8 thereof, proline residues as amino acid residues corresponding to positions 4 and 6 thereof, and a glycine residue as an amino acid residue corresponding to position 9 thereof.

(10) The plant according to (9), wherein the amino acid sequence of the peptide (b) further has a valine residue, an isoleucine residue, or a proline residue as an amino acid residue corresponding to position 3 of the amino acid sequence represented by SEQ ID NO: 1, an isoleucine residue or a valine residue as an amino acid residue corresponding to position 5 thereof, a leucine residue or a phenylalanine residue as an amino acid residue corresponding to position 7 thereof, and/or a valine residue as an amino acid residue corresponding to position 8 thereof.

(11) The plant according to (9) or (10), wherein the amino acid sequence of the peptide (b) has an alanine residue as an amino acid residue corresponding to position 2 of the amino acid sequence represented by SEQ ID NO: 1, a valine residue as an amino acid residue corresponding to position 3 thereof, an isoleucine residue as an amino acid residue corresponding to position 5 thereof, and/or a leucine residue as an amino acid residue corresponding to position 7 thereof.

(12) The plant according to (9), wherein the peptide (b) consists of any of the amino acid sequences represented by SEQ ID NOs: 2 to 7.

(13) The plant according to (9), wherein the peptide (b) consists of any of the amino acid sequences represented by SEQ ID NOs: 8 to 18.

(14) The plant according to any of (9) to (13), wherein the nucleic acid expression system overexpresses the nucleic acid incorporated therein.

(15) The plant according to any of (9) to (14), wherein the nucleic acid expression system constitutively expresses the nucleic acid incorporated therein.

(16) The plant according to any of (9) to (14), wherein the nucleic acid expression system inducibly expresses the nucleic acid incorporated therein.

(17) The plant according to any of (9) to (16), wherein the nucleic acid expression system is an expression vector.

(18) Progeny of a plant according to any of (9) to (17).

The present specification encompasses the contents described in the specification and/or drawings of Japanese Patent Application No. 2011-024394 on which the priority of the present application is based.

### Advantageous Effects of Invention

The composition of the present invention can be applied to a plant to thereby confer activities of augmenting disease resistance and/or branching to the plant.

The composition of the present invention comprising a chemically synthesized active peptide as an active ingredient can provide an inexpensive plant disease resistance-augmenting agent and a method for preventing and treating infectious disease in a plant using the same.

The composition of the present invention can control the number of branching in a plant and can enhance the yields of agricultural or horticultural crops.

### BRIEF DESCRIPTION OF DRAWINGS

FIG. 1 shows the full-length amino acid sequence of Arabidopsis thaliana BIL3 and its characteristic domain. As shown in this diagram, BIL3 has an N-terminal extracellular, a C-terminal active domain (indicated by italicized letters) that is extracellularly secreted and functions as a peptide hormone, and a cleavage site therebetween for cleaving the extracellular transport signal after secretion.

FIG. 2 shows the amino acid sequence alignment of BIL3 and its paralogs and orthologs, and an active domain (peptide hormone) cleaved off from each protein after processing. In each amino acid sequence, “-” represents a gap, and “X” represents an undetermined amino acid residue. Numbers in the active domain represent the position of each amino acid residue counted from a threonine residue located at the N terminus of the active domain of Arabidopsis thaliana (A. thaliana) BIL3 as position 1. The active domain of each paralog or ortholog, when aligned as shown in this diagram, exhibits the greatest degree of matching with amino acids in the active domain of Arabidopsis thaliana BIL3. Thus, the position of the active domain in each paralog or ortholog corresponds to the position of amino acid residues (shown in this diagram) defined in the active domain of BIL3 described above.

FIG. 3 morphologically shows the aerial parts of a wild-type strain and a bil3 mutant. As is evident from this diagram, the number of flower stalks and the number of branches were increased in the bil3 mutant.

FIG. 4 is a diagram statistically showing the number of flower stalks (FIG. 4A) and the number of branches (FIG. 4B) in the wild-type strain and the bil3 mutant shown in FIG. 3.

FIG. 5 shows the expression level of the BIL3 gene in the wild-type strain and the bil3 mutant. In this diagram, the expression of the BIL3 gene is indicated by relative value with the expression level of the BIL3 gene in the wild-type strain detected by real-time RT-PCR as 1.

FIG. 6 shows the expression level of the BIL3 gene in the wild-type strain and a 35S::BIL3 transformant. In this diagram, the expression of the BIL3 gene is indicated by relative value with the expression level of the BIL3 gene in the wild-type strain detected by real-time RT-PCR as 1.

FIG. 7 shows the expression level of the PR1 gene in the wild-type strain and the bil3 mutant. In this diagram, the expression of the PR1 gene is indicated by relative value with the expression level of the PR1 gene in the wild-type strain detected by real-time RT-PCR as 1.

### DESCRIPTION OF EMBODIMENTS

Hereinafter, embodiments of the present invention will be described specifically.

1. Peptide Augmenting Plant Disease Resistance and/or Branching, or Salt Thereof

1-1. Summary and Constitution

The first embodiment of the present invention relates to a peptide or a salt thereof. The peptide of the present embodiment or the salt thereof has activities of augmenting plant disease resistance and/or branching.

In the present specification, the “plant” corresponds to bryophytes, ferns, angiosperms, and gymnosperms. The angiosperms encompass both dicot and monocot plants. The plant also includes both herbs and arbores. In the present invention, particularly preferred examples of the plant include agriculturally or commercially important plants, for example, crop plants such as cereals, vegetables, fruits, and garden flowers. Specific examples of the plant include: monocot plants such as plants of the family Poaceae (including rice, wheat, barley, rye, oat, pearl barley (Coix lacryma-jobi var. ma-yuen), millet (Panicum miliaceum), Italian millet (Setaria italica), Japanese millet (Echinochloa esculenta), Finger millet (Eleusine coracana (Linn.) Gaertn.), corn, Indian millet (Sorghum bicolor), kaoliang, sorghum (Sorghum vulgare Pers.), sugarcane, bamboo, and bamboo grass) and the family Zingiberaceae (including ginger, myoga ginger (Zingiber mioga), and turmeric); and dicot plants such as plants of the family Solanaceae (including tobacco, tomato, eggplant, bell pepper, chili pepper, and petunia), the family Leguminosae (including soybean, peanut, azuki bean, green pea, common bean, lentil, pea, broad bean, kuzu vine, sweet pea, and tamarind), the family Rosaceae (including strawberry, rose, Japanese apricot, cherry, apple, pear, peach, loquat, almond, plum, flowering quince, and Japanese kerria), the family Cucurbitaceae (including cucumber, balsam apple, gourd, pumpkin, melon, watermelon, luffa, and bottle gourd (Lagenaria siceraria var. gourda)), the family Liliaceae (including lily, tulip, hyacinth, lily of the valley, asparagus, Welsh onion, and onion), the family Brassicaceae (including lettuce, cabbage, radish, Chinese cabbage, turnip, and oilseed rape), the family Vitaceae, the family Rutaceae, the family Malvaceae (including cotton, okra, Chinese mallow (Malva verticillata), and rose of Sharon (Hibiscus syriacus)), the family Primulaceae (including cyclamen), the family Theaceae (including tea plant), the family Moraceae (including fig and mulberry tree), the family Actinidiaceae (including kiwi fruit), the family Anacardiaceae (including pistachio and mango), the family Piperaceae, and the family Ericaceae (including rhododendron, satsuki azalea (Rhododendron indicum), azalea, erica, and Belgium azalea (Rhododendron simsii cv))). The “plant” described in the present specification is not limited to a plant body and encompasses all of plant cells, tissues, and organs (embryos, meristems, seeds, shoots, roots, stems, leaves, and flowers).

In the present specification, the “disease” or the “plant disease” refers to a disease in a plant caused by a pathogen. In this context, the “pathogen” refers to any of agents, such as viroids, viruses, phytoplasmas, bacteria, fungi (including yeasts, molds, and basidiomycetes), slime molds, protozoans, or nematodes, which are infective to plants and bring about some pathological symptom to the plants through their infection. In the present invention, the pathogen preferably corresponds to, particularly, viroids, viruses, phytoplasmas, and bacteria. Examples thereof include microbes of the genera Bacillus, Aspergillus, Penicillium, Schizosaccharomyces, Paenibacillus, and Trichoderma.

In the present specification, the “disease resistance” refers to the effect of preventing or suppressing the pathogenic infection or the onset of a pathological symptom caused thereby. This effect is controlled by the natural immune system of a plant. Thus, the phrase “augmenting disease resistance” refers to the prevention or suppression of the pathogenic infection or the onset of a pathological symptom caused thereby by more potentiating the natural immune system of a plant.

In the present specification, the “branching” or the “plant branching” refers to the development and outgrowth of a lateral bud or an auxiliary bud serving as an apex from which a new shoot grows from the stem (including flower stalks), trunk, or branch of a plant. The branching of the present invention also encompasses “tillers”, which are new lateral buds developed and grown from a part near the root as found in monocot plants, etc. In the present specification, the number of branches or stems (including flower stalks) developed by the “branching” is referred to as the “number of branching”.

In the present specification, the phrase “augmenting branching” refers to the activation of branching of a plant in the process of growth of the plant. The plant with augmented branching has the increased number of branching and also has an increased weight ratio compared with common plant bodies of the same species.

1-1-1. Peptide

In the present specification, the simply described “peptide” means a molecule containing two or more amino acids linked via an amide bond. Thus, the term “peptide” encompasses both oligopeptides and polypeptides. The term “oligopeptide” means a peptide consisting of 20 or less amino acid residues. The term “polypeptide” means a peptide consisting of 21 or more amino acid residues.

The amino acids constituting the peptide of the present invention may be in any of D, L, and DL forms (racemates). Particularly, an L form is preferred. The amino acids constituting the peptide of the present invention derived from a natural protein are all in an L form. In the case of preparing the peptide of the present invention by chemical synthesis, the peptide may consist of only L-amino acids or only D-amino acids or L-amino acid(s) and D-amino acid(s) in combination.

The “peptide having activities of augmenting plant disease resistance and/or augmenting plant branching” (hereinafter, referred to as an “active peptide”) refers to only an active domain (which corresponds to positions 53 to 61 of SEQ ID NO: 8), which is a region extracellularly secreted as a peptide hormone in BIL3 protein or its paralogs or orthologs, or refers to a peptide comprising the active domain. The active peptide may have any number of amino acids added to the N terminus and/or C terminus of the active domain as long as the active domain has the activities described above. Such amino acids may be amino acids naturally adjacent to the active domain or may be amino acids that are not naturally adjacent to the active domain.

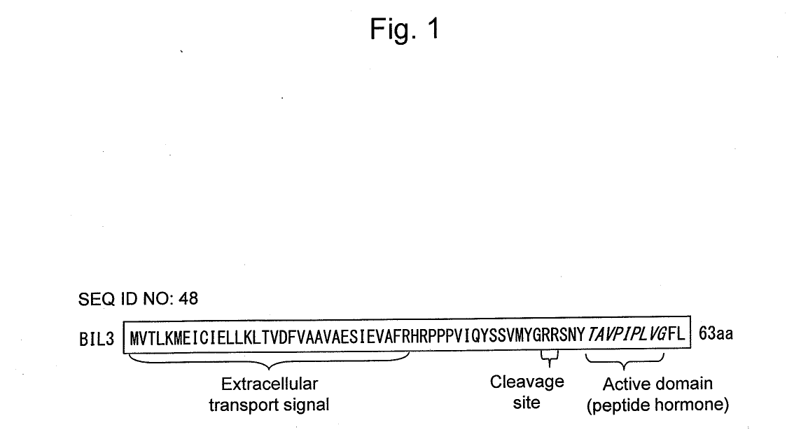

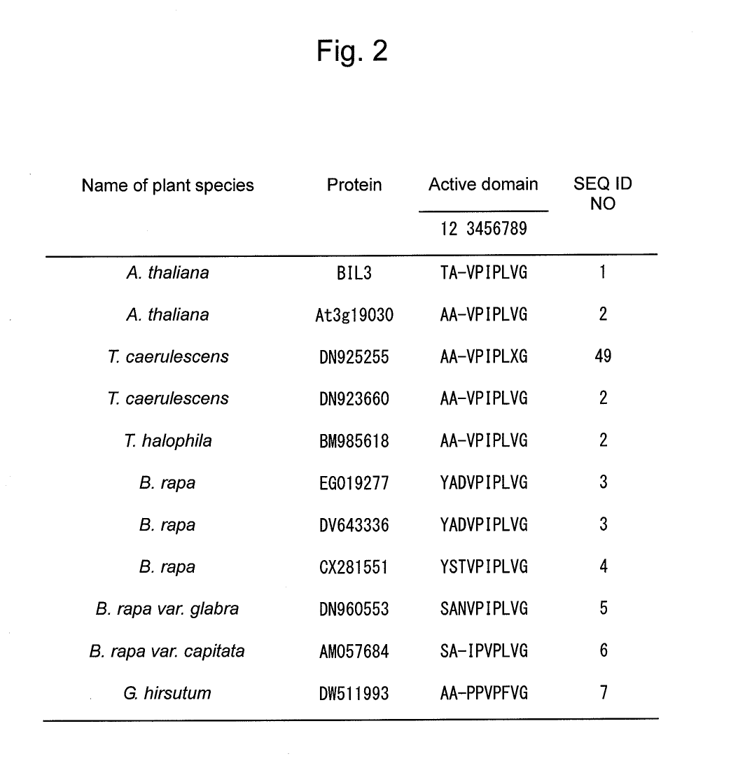

The protein “BIL3 (Brz-insensitive-long hypocotyl 3)” is encoded by Arabidopsis thaliana BIL3 gene. BIL3 is an extracellular secretion-type protein that has a full length of 63 amino acids represented by SEQ ID NO: 8 (NCBI-ID No. At1g49500) and has an N-terminal extracellular transport signal (secretory signal; positions 1 to 32 of SEQ ID NO: 8), a C-terminal active domain (see FIG. 2; positions 53 to 61 of SEQ ID NO: 8) that is highly conserved across species, and a cleavage site (positions 48 to 49 of SEQ ID NO: 8) therebetween (see FIG. 1). BIL3 is cleaved at the cleavage site after translation to remove the extracellular transport signal peptide. The active domain undergoes N-terminal processing and C-terminal processing and is then extracellularly secreted as a peptide hormone. Similar processing of active peptides has been reported as to, for example, Arabidopsis thaliana PSY1 (Amano Y. et al., (2007) Proc. Natl. Acad. Sci. USA, 46: 18333-18338). Thus, the active domain of BIL3 is presumably composed of the sequence of 9 amino acids represented by SEQ ID NO: 1.

BIL3 has three Arabidopsis thaliana paralogs (NCBI-ID No. At3g19030, NCBI-ID No. At4g33960, and NCBI-ID No. At2g15830) consisting of the amino acid sequences represented by SEQ ID NOs: 9, 34, and 37, respectively, and also has BIL3 orthologs derived from other plant species. Specific examples of the BIL3 orthologs include proteins of Thlaspi caerulescens of the family Brassicaceae consisting of the amino acid sequences represented by SEQ ID NO: 10 (NCBI-ID No. DN925255) and SEQ ID NO: 11 (NCBI-ID No. DN923660), proteins of Thellungiella halophila consisting of the amino acid sequences represented by SEQ ID NO: 12 (NCBI-ID No. BM985618) and SEQ ID NO: 36 (NCBI-ID No. DN779022), proteins of Brassica rapa (oilseed rape) consisting of the amino acid sequences represented by SEQ ID NO: 13 (NCBI-ID No. EG019277), SEQ ID NO: 14 (NCBI-ID No. DV643336), SEQ ID NO: 15 (NCBI-ID No. CX281551), and SEQ ID NO: 35 (NCBI-ID No. CN727308), a protein of Brassica rapa var. glabra (Chinese cabbage) consisting of the amino acid sequence represented by SEQ ID NO: 16 (NCBI-ID No. DN960533), a protein of Brassica oleracea var. capitata (cabbage) consisting of the amino acid sequence represented by SEQ ID NO: 17 (NCBI-ID No. AM057684), and a protein of Gossypium hirsutum (upland cotton) of the family Malvaceae consisting of the amino acid sequence represented by SEQ ID NO: 18 (NCBI-ID No. DW511993). All of these BIL3 paralogs and orthologs have an N-terminal extracellular transport signal, a C-terminal active domain, and a cleavage site therebetween.

The “activities of augmenting plant disease resistance and/or augmenting plant branching” are activities possessed by, for example, the active domain of BIL3 or the active domain of each BIL3 paralog or ortholog. Specific examples of such domains include (A) the active domain of BIL3 consisting of the amino acid sequence represented by SEQ ID NO: 1 and (B) the active domain of a BIL3 paralog or ortholog that consists of an amino acid sequence having identity to 4 or more amino acids in the amino acid sequence represented by SEQ ID NO: 1 and having an alanine residue or a serine residue as an amino acid residue corresponding to position 2 of the amino acid sequence represented by SEQ ID NO: 1, nonpolar amino acid residues as amino acid residues corresponding to positions 3, 5, 7, and 8 thereof, proline residues as amino acid residues corresponding to positions 4 and 6 thereof, and a glycine residue as an amino acid residue corresponding to position 9 thereof.

The “amino acid identity” of the active domain (B) refers to the number of identical amino acid residues between the amino acid sequences of the active domain of BIL3 and the active domain of the BIL3 paralog or ortholog to be compared when the amino acid sequences are aligned with a gap introduced, if necessary, in one or both of the amino acid sequences so as to give the highest degree of matching between their amino acid residues. The amino acid identity can be preferably 4 or more amino acids (44% or higher), more preferably 5 or more amino acids (55% higher), even more preferably 6 or more amino acids (66% or higher), with respect to the amino acid sequence (9 amino acids) of the BIL3 active domain. In this context, “%” refers to the ratio (%) of the number of identical (to the amino acid residues in the amino acid sequence of BIL3) amino acid residues with the greatest degree of matching in the amino acid sequence to be compared to the total number of amino acid residues in the amino acid sequence of BIL3. The % identity can be determined easily using a program known in the art, such as a homology search program BLAST Search (http://www.ncbi.nlm.nih.gov/blast/Blast.cgi). The “gap” refers to 1 or several amino acid residue(s). In this context, the term “several” corresponds to 2 to 5, 2 to 4, or 2 to 3 amino acids. Specifically, the active domain (B), when aligned with the BIL3 active domain, may have the deletion or addition of 1 or several amino acid residue(s) compared with the BIL3 active domain.

In the active domain (B), the phrase “corresponding to position X of the amino acid sequence (represented by SEQ ID NO: 1)” (wherein X represents any number of 1 to 9) defines the position of an amino acid residue in the active domain (B) with respect to the amino acid sequence of the BIL3 active domain. Specifically, the positions of amino acid residues in the BIL3 active domain represented by SEQ ID NO: 1 are numbered 1 to 9 in order from the N terminus. Subsequently, the BIL3 active domain and the active domain (B) are aligned so as to give the highest degree of matching between their amino acid residues. In this respect, the phrase described above represents the position of an amino acid residue in the active domain (B) corresponding to a position in the BIL3 active domain. In this context, the “corresponding amino acid residue” is not necessarily required to be an amino acid residue identical to that in the BIL3 active domain. Preferably, this amino acid residue is an identical amino acid residue or a similar amino acid residue. In this context, the “similar amino acid” refers to an amino acid that belongs to the same group of amino acids classified on the basis of properties such as electric charges, side chains, polarity, and aromatic properties. Examples of such groups include a basic amino acid group (arginine, lysine, and histidine), an acidic amino acid group (aspartic acid and glutamic acid), a nonpolar amino acid group (glycine, alanine, phenylalanine, valine, leucine, isoleucine, proline, methionine, and tryptophan), a polar uncharged amino acid group (serine, threonine, asparagine, glutamine, tyrosine, and cysteine), a branched amino acid group (leucine, isoleucine, and valine), an aromatic amino acid group (phenylalanine and tyrosine), a heterocyclic amino acid group (histidine, tryptophan, and proline), and an aliphatic amino acid group (glycine, alanine, leucine, isoleucine, and valine).

The “amino acid residue corresponding” to a certain position in the amino acid sequence of the BIL3 active domain may be absent, and/or an amino acid residue present in the amino acid sequence of the active domain (B) may not correspond to an amino acid residue in the BIL3 active domain. Such a case corresponds to, for example, the case where 1 or several amino acid residue(s) are deleted or added when the BIL3 active domain and the active domain (B) are aligned as described above.

Preferably, the active domain (B) has a valine residue as an amino acid residue corresponding to position 3 of the amino acid sequence represented by SEQ ID NO: 1, an isoleucine residue as an amino acid residue corresponding to position 5 thereof, a leucine residue as an amino acid residue corresponding to position 7 thereof, and/or a valine residue as an amino acid residue corresponding to position 8 thereof. Specific examples of such active domains include active domains consisting of the amino acid sequences represented by SEQ ID NOs: 2 to 7.

The active domain may consists of an amino acid sequence represented by any of SEQ ID NOs: 30 to 33.

The peptide of the present embodiment consists of an active peptide comprising the active domains (A) and/or (B). The length of the active peptide is not limited as long as the active peptide retains the activities. In consideration of a peptide hormone as the active domain contained therein or the chemical synthesis of the active peptide, the active peptide is desirably a short-chain peptide. The length of the active peptide is preferably 100 or less amino acids, more preferably 70 or less amino acids, even more preferably 50 or less amino acids, further preferably 30 or less amino acids, further preferably 20 or less amino acids. Such an active peptide corresponds to, for example, an active peptide further having several amino acids added to the N terminus and/or the C terminus of the active domain. The active peptide is most preferably the active domain itself, which is the minimum unit capable of functioning as a peptide hormone. Hence, the length of the active peptide is most preferably the number of amino acid residues in the active domain, for example, 9 or 10 amino acids in the case of the active domains represented by SEQ ID NOs: 1 to 7.

The peptide comprising the active domains (A) and/or (B) may be modified as long as the resulting peptide has the activities of augmenting plant disease resistance and/or plant branching. The modification includes modification with a labeling material as well as glycosylation, acetylation, formylation, amidation, phosphorylation, and PEGylation.

The same labeling material as that described in Embodiment 1 can be used as the labeling material. The modification with the labeling material is useful in detecting the anti-marker antibody of the present embodiment and its antigen binding fragment described later.

The modification regarding glycosylation may be natural glycosylation or may occur at a modified glycosylation site obtained by modifying a natural glycosylation site by a recombinant DNA technique or chemical treatment. The glycosylation site can be modified by any method known by those skilled in the art. Examples of the method include a method based on the gene manipulation as mentioned above, a method using glycosylation mutants, a method based on coexpression with one or more enzyme(s), for example, DI N-acetylglucosamine transferase III (GnTIII), and a method involving causing the expression of the peptide in various organisms or cell lines derived from various organisms and purifying the peptide, followed by sugar chain modification. For the method for preparing a modified glycosylation site by gene manipulation, see, for example, Umana et al., 1999, Nat. Biotechnol 17: 176-180; Davies et al., 2001, Biotechnol Bioeng 74: 288-294; Shields et al., 2002, J Biol Chem 277: 26733-26740; and Shinkawa et al., 2003, J Biol Chem 278: 3466-3473. For the method for modifying a sugar chain, see, for example, U.S. Pat. No. 6,218,149; European Patent No. 0,359,09681; U.S. Patent Publication No. 2002/0028486; International Publication No. WO 03/035835; U.S. Patent Publication No. 2003/0115614; U.S. Pat. No. 6,218,149; and U.S. Pat. No. 6,472,511. The modification by PEGylation involves binding a water-soluble polymer molecule such as polyethylene glycol (PEG) to the peptide serving as an active ingredient. The PEGylation can be achieved by chemically binding PEG to the N-terminal amino group, C-terminal carboxyl group, or lysine (Lys) residue ε-amino group of the antibody, etc. The peptide modified by the PEGylation can have an enhanced in vivo half-life.

The active peptide of the present embodiment can be synthesized according to, for example, a chemical synthesis method such as a fluorenylmethyloxycarbonyl (Fmoc) or t-butyloxycarbonyl (tBoc) method (Lectures on Biochemical Experiments —1—, Chemistry of Proteins —IV—, Chemical Modification and Peptide Synthesis, The Japanese Biochemical Society ed., Tokyo Kagaku Dojin Co., Ltd. (Japan), 1981). Alternatively, the active peptide of the present invention may be synthesized by a method known in the art using various commercially available peptide synthesizers (e.g., PSSM8 manufactured by Shimadzu Corp. and Model 433A manufactured by Applied Biosystems, Inc. (ABI)). Alternatively, a nucleic acid encoding the active peptide can be prepared using a genetic engineering approach known in the art (see e.g., Sambrook, J. et al., (1989) Molecular Cloning: a Laboratory Manual Second Ed., Cold Spring Harbor Laboratory Press, Cold Spring Harbor, N.Y.) and then incorporated into an expression vector, which is in turn introduced into host cells to produce the active peptide of interest in the host cells. In the case of biosynthesizing the active peptide by a genetic engineering approach, the nucleic acid encoding the peptide is linked to a nucleic acid encoding an extracellular transport signal. This approach is convenient because the active peptide of interest can be secreted extracellularly from the host cells after protein expression and collected easily from a culture supernatant thereof. Alternatively, the culture supernatant or a culture solution containing the host cells may be used in itself as the composition of the second embodiment described later without collecting or purifying the active peptide.

1-1-2. Salt of Peptide

The salt of the peptide of the present embodiment refers to a salt of the peptide described in the paragraph “1-1-1. Peptide”. In this context, the “salt” can be any agriculturally acceptable salt without particular limitations. Examples thereof include acid-addition salts and base-addition salts. Examples of the acid-addition salts include: salts with inorganic acids such as hydrochloric acid, sulfuric acid, nitric acid, and phosphoric acid; and salts with organic acids such as acetic acid, malic acid, succinic acid, tartaric acid, and citric acid. Examples of the base-addition salts include: salts with alkali metals such as sodium and potassium; salts with alkaline earth metals such as calcium and magnesium; and salts with ammonium and amines such as triethylamine.

1-2. Advantage

The peptide of the present invention or the salt thereof can be applied to a plant to thereby confer activities of augmenting disease resistance and/or branching to the plant.

The peptide of the present invention or the salt thereof is rapidly decomposed in the natural world and as such, has the advantage that the peptide of the present invention or the salt thereof, even when used as an agricultural chemical, does not remain over a long period in soil, water, or plant bodies and has only small influence on environments or animals including humans.

The active domain that is responsible for the activities of the peptide of the present invention or the salt thereof is not a special substance such as alkaloid expressed only in particular plant species but a general plant peptide hormone also found in vegetables routinely used such as cabbage or Chinese cabbage and as such, is very safe.

The peptide of the present invention or the salt thereof even having around 10 amino acids has activities and as such, can be synthesized inexpensively in large amounts by chemical synthesis. Hence, the peptide of the present invention or the salt thereof can serve as an active ingredient, instead of expensive BR, in a plant disease resistance-augmenting agent and a plant disease-resistant composition.

The peptide of the present invention or the salt thereof can control plant branching. For example, the peptide of the present invention or the salt thereof can augment branching to increase the number of branching, particularly, the number of flower stalks, thereby increasing the yields of agricultural or horticultural crops. The peptide of the present invention or the salt thereof can enhance the weight ratios of plant bodies compared with wild-type strains grown for the same period in the same environment thereas. As a result, a plant biomass can be increased. Thus, the productivity of forestry, bioethanol, etc., can also be improved.

2. Composition

2-1. Summary and Constitution

The second embodiment of the present invention relates to a composition. The composition of the present embodiment comprises at least one peptide having activities of augmenting plant disease resistance and/or augmenting plant branching as an active ingredient thereof and has activities of augmenting plant disease resistance and/or branching.

2-1-1. Active Ingredient

The composition of the present embodiment contains at least one active peptide described in the first embodiment as an active ingredient. The composition may contain two or more peptides. In such a case, the types of these peptides may be derived from the same organism species or may be a combination derived from different organism species. Also, these two or more peptides contained in the composition may have the same lengths or may have different lengths.

The amount of the active peptide contained in the composition of the present embodiment depends on conditions such as the type of the active peptide, the strength of its activities, the type of a carrier contained therein, the type of a recipient plant, intended application, an application method, and the type of a drug, if contained, having other pharmacological effects. The content thereof can be determined appropriately in consideration of conditions under which the composition applied to a target plant can augment disease resistance and/or branching in the plant.

2-1-2. Agriculturally Acceptable Carrier

The composition of the present invention may also comprise, if necessary, an agriculturally acceptable carrier. The “agriculturally acceptable carrier” refers to a substance that facilitates the application of the composition to a plant and suppresses the decomposition of the active peptide serving as an active ingredient or/and controls the rate at which the active peptide acts. Examples of the agriculturally acceptable carrier include solvents and auxiliaries.

Examples of the “solvents” include water, aromatic compound solvents (e.g., benzene, toluene, xylene, tetrahydronaphthalene, alkylated naphthalene, and derivatives thereof), paraffins (e.g., mineral oil fractions), chloroform, carbon tetrachloride, ketones (e.g., acetone and cyclohexanone), pyrrolidones (e.g., NMP and NOP), acetate (glycol diacetate), glycols, aliphatic dimethylamides, fatty acids, fatty acid esters, and mixed solvents thereof. Alternatively, a medium for cell culture or microbial culture may be used.

Preferred examples of the auxiliaries include natural mineral powders, synthetic mineral powders, emulsifiers, dispersants, and surfactants.

The “natural mineral powders” correspond to, for example, kaolin, clay, talc, and chalk.

The “synthetic mineral powders” correspond to, for example, highly dispersible silica and silicate.

The “emulsifiers” correspond to nonionic emulsifiers and anionic emulsifiers (e.g., polyoxyethylene fatty alcohol ether, alkyl sulfonate, and aryl sulfonate).

Examples of the “dispersants” include lignosulfite waste liquors and methylcellulose.

The “surfactants” correspond to, for example, alkali metal salts, alkaline earth metal salts, and ammonium salts of lignosulfonic acid, naphthalenesulfonic acid, phenolsulfonic acid, and dibutylnaphthalenesulfonic acid, alkylaryl sulfonate, alkyl sulfate, alkyl sulfonate, fatty alcohol sulfate, fatty acid and sulfated fatty alcohol glycol ethers, condensates of sulfonated naphthalene or a naphthalene derivative and formaldehyde, condensates of naphthalene or naphthalenesulfonic acid, phenol, and formaldehyde, polyoxyethylene octyl phenyl ether, ethoxylated isooctylphenol, octylphenol, nonylphenol, alkylphenyl polyglycol ether, tributylphenyl polyglycol ether, tristearylphenyl polyglycol ether, alkylaryl polyether alcohol, condensates of alcohol or fatty alcohol and ethylene oxide, ethoxylated castor oil, polyoxyethylene alkyl ether, ethoxylated polyoxypropylene, lauryl alcohol polyglycol ether acetal, sorbitol ester, lignosulfite waste liquors, and methylcellulose.

Preferably, the carrier for use in the composition of the present embodiment has no harmful effect or only small influence on environments such as soil and water quality or on animals, particularly, humans, when applied to the plant of interest.

The composition of the present embodiment may contain one or more agriculturally acceptable carrier(s) described above. The composition of the present invention may further contain an additional disease resistance-augmenting agent and/or an additional branching-augmenting agent, and an active ingredient having other pharmacological effects, for example, a pesticide, an insecticide, a herbicide, a bactericide, or a fertilizer (e.g., urea, ammonium nitrate, and superphosphate), without influencing the effects or advantages of the branching-suppressing agent of the present invention.

The dosage form of the composition of the present embodiment may be any of a liquid form, a solid (including semisolid) form, and a combination thereof. The dosage form can be set to a conventional pharmaceutical form, for example, solutions, oily dispersions, emulsions, suspensions, dusts, powders, pastes, gels, pellets, tablets, and granules, which permit direct spraying, coating, and/or dipping.

3. Method for Suppressing Microbial Infection

The third embodiment of the present invention relates to a method for suppressing microbial infection. The method for suppressing microbial infection according to the present embodiment activates the disease resistance of the plant of interest to prevent or suppress infection from a plant-infective microbe. In this context, the “plant-infective microbe” refers to a microbe that is infective to a plant and brings about some pathological symptom to the host plant through its infection.

The method for suppressing microbial infection according to the present embodiment comprises an action step. Hereinafter, this step will be described specifically.

3-1. Action Step

The “action step” of the present embodiment is the step of allowing the peptide (i.e., active peptide) of the first embodiment and/or the composition of the second embodiment to act on a recipient plant. In this context, the phrase “acting on a plant” means that the active peptide and/or the composition are contacted with the recipient plant to cause the plant body to incorporate therein the active peptide serving as an active ingredient. The method for contacting the active peptide of the first embodiment and/or the composition of the second embodiment is not limited. The method can be selected appropriately according to a contact site with the recipient plant body.

For example, the active peptide of the first embodiment and/or the composition of the second embodiment may be contacted with the aerial part of the recipient plant body. In such a case, examples of the contact method include methods such as the nebulization, spraying, coating, injection, dipping, and wound inoculation (including needle prick inoculation) of the active peptide and/or the composition. Since the active peptide serving as an active ingredient is based on the peptide hormone, the active peptide and/or the composition can be absorbed easily from the surface of the plant body by any of these contact methods. In this case, the dosage form of the active peptide of the first embodiment and/or the composition of the second embodiment is preferably a liquid, powdery, or gel solid form. The contact site on the aerial part may be any desired area such as stems or the base of the petiole without particular limitations. The contact site may be a portion or the whole of the plant body and is preferably a site most commonly found in the route of infection of the recipient plant with the plant-infective microbe. Examples thereof include leaves and stems.

Alternatively, the active peptide of the first embodiment and/or the composition of the second embodiment may be contacted with the root of the recipient plant body. In this case, the active peptide of the first embodiment and/or the composition of the second embodiment is absorbed from the root and thereby spread throughout the plant body where the active ingredient can then exert the effects of the present invention. For example, for hydroponic cultivation, the contact method specifically involves applying the active peptide of the first embodiment and/or the composition of the second embodiment to the plant by addition into a hydroponic solution. This approach has the advantages that: the concentration of the active peptide serving as an active ingredient can be controlled in the hydroponic solution; the decomposition of the active peptide can be suppressed by sterilizing the hydroponic solution; etc.

Alternatively, the plant disease resistance-augmenting agent may be applied directly or indirectly into or onto soil. This application method is convenient when the agent is applied to a wide area such as an agricultural field. However, note that the active peptide serving as the active ingredient of the present invention tends to be decomposed in a relatively short time by the action of microbes or the like in soil. If short-term effects are desired by the transient action of the active peptide of the first embodiment and/or the composition of the second embodiment, the active peptide and/or the composition can be applied directly into soil. Alternatively, the composition of the second embodiment comprising the active peptide of the first embodiment enclosed in a sustained-release inclusion body may be applied indirectly to the plant of interest. This approach is also convenient because the active peptide in the composition is absorbed from the root of the recipient plant by escaping decomposition in soil and thereby spread throughout the plant body where the active ingredient can then exert the effects of the present invention in each cell.

The amount of the active peptide of the first embodiment and/or the composition of the second embodiment applied varies depending on the type of the active peptide (in the case of the composition of the second embodiment, the type of the active peptide contained therein) or the type of the recipient plant. The amount of the active peptide of the first embodiment and/or the composition of the second embodiment applied even to recipient plants of the same species varies between hydroponic cultivation and soil cultivation. This is because the active peptide serving as an active ingredient is generally decomposed by the action of microbial decomposition in soil at a faster rate than that in a hydroponic solution. Thus, the amount of the active peptide and/or the composition applied can be determined appropriately according to situations, purposes, and needs by those skilled in the art.

3-2. Advantage

The method for suppressing microbial infection according to the present embodiment can apply the peptide of the first embodiment and/or the composition of the second embodiment to the desired plant to thereby confer disease resistance to the plant.

The method for suppressing microbial infection according to the present embodiment can produce the effects of the active peptide of the first embodiment and/or the active ingredient in the composition of the second embodiment by contact with a plant body because the active peptide and/or the active ingredient can act as a peptide hormone. Hence, the method of the present invention does not require preparing the desired plant into a transgenic plant for conferring disease resistance thereto and can be applied even to edible crops (vegetables, cereals, and fruits).

4. Method for Augmenting Plant Branching

The fourth embodiment of the present invention relates to a method for augmenting plant branching. The method for augmenting plant branching according to the present embodiment comprises an action step. Hereinafter, this step will be described specifically.

4-1. Action Step

The “action step” of the present embodiment is the step of allowing the active peptide of the first embodiment and/or the composition of the second embodiment to act on a recipient plant. The present step is basically similar to the action step of the method for suppressing microbial infection according to the third embodiment, and a specific method therefor follows the step, as a rule. Hence, only a point different from the action step of the method for suppressing microbial infection according to the third embodiment will be described below.

The method for augmenting plant branching according to the present embodiment differs from the method of the preceding embodiment in that the method of the present embodiment is aimed at allowing the active peptide of the first embodiment and/or the composition of the second embodiment to act on a recipient plant to activate the branching of the plant, resulting in increase in the number of branching. Of course, the peptide of the first embodiment and/or the active peptide serving as the active ingredient of the composition described in the second embodiment can augment the branching of the recipient plant and can also confer disease resistance to the plant. Thus, these effects can be achieved at the same time by the application of the active peptide of the first embodiment and/or the composition of the second embodiment.

Also in the action step of the present embodiment, a contact site on the plant body with the active peptide and/or the composition is not limited. Preferably, the active peptide and/or the composition is contacted directly with a site where cell differentiation or growth is active, in consideration of obtained results showing that the BIL3 gene is expressed in shoot apices, whole roots, and veins and the fact that plant branching generally occurs in shoot apices or the base of the petiole.

4-2. Advantage

The method for augmenting plant branching according to the present embodiment can apply the active peptide of the first embodiment and/or the composition of the second embodiment to the desired plant to activate the branching of the plant, thereby increasing the number of branching. The method of the present embodiment can also increase the number of flower stalks and as such, can provide a method for enhancing the yields of horticultural or agricultural crops by increasing the number of flower buds.

The active peptide and/or the active ingredient in the composition for use in the method of the present embodiment, as in the method for suppressing microbial infection according to the third embodiment, are found in general plants and are spontaneously and highly decomposable. Thus, the active peptide and/or the active ingredient are highly safe to human bodies and have only small influence on the natural world by application.

5. Transgenic Plant

5-1. Summary and Constitution

The fifth embodiment of the present invention relates to a transgenic plant with augmented plant disease resistance and/or branching activity.

The “transgenic plant” refers to a transformed plant expressibly containing gene(s) derived from the same species and/or different species as or from the plant as an exogenous gene. In the present specification, the “transgenic plant” particularly refers to a transformed plant intracellularly comprising an exogenous nucleic acid expression system comprising, in an expressible state, a nucleic acid encoding the active peptide described in the first embodiment, whereby the disease resistance and/or branching activity of the plant is augmented compared with wild-type strains.

5-1-1. Nucleic Acid

The nucleic acid encoding the peptide serving as the active ingredient described in the first embodiment will be described.

The “nucleic acid encoding the peptide serving as the active ingredient described in the first embodiment” refers to a nucleic acid encoding the active peptide described in the first embodiment. In the present invention, the term “nucleic acid” mainly refers to natural nucleic acids such as DNA and/or RNA and can also include artificially chemically modified or constructed nucleic acids or nucleic acid analogs. If necessary, the nucleic acid may be labeled, at its phosphate group, sugar moiety, and/or base moiety, with a nucleic acid-labeling material.

As described above, the active peptide comprises an active domain as the central active site of the active ingredient. This active domain corresponds to a peptide hormone which is a mature form of the Arabidopsis thaliana BIL3 protein or its paralog or ortholog. Specific examples of the nucleic acid include a nucleic acid comprising a nucleotide sequence encoding the active domain of BIL3 consisting of the amino acid sequence represented by SEQ ID NO: 1. Also, the nucleic acid refers to a nucleic acid encoding the active domain in each BIL3 paralog or ortholog, i.e., a nucleic acid encoding a peptide that comprises an amino acid sequence having identity to 4 or more amino acids in the amino acid sequence represented by SEQ ID NO: 1 and having an alanine residue or a serine residue as an amino acid residue corresponding to position 2 of the amino acid sequence represented by SEQ ID NO: 1, nonpolar amino acid residues as amino acid residues corresponding to positions 3, 5, 7, and 8 thereof, proline residues as amino acid residues corresponding to positions 4 and 6 thereof, and a glycine residue as an amino acid residue corresponding to position 9 thereof. Preferable examples of such nucleic acids include a nucleic acid encoding the peptide (b) that comprises an amino acid sequence further having a valine residue, an isoleucine residue, or a proline residue as an amino acid residue corresponding to position 3 of the amino acid sequence represented by SEQ ID NO: 1, an isoleucine residue or a valine residue as an amino acid residue corresponding to position 5 thereof, a leucine residue or a phenylalanine residue as an amino acid residue corresponding to position 7 thereof, and/or a valine residue as an amino acid residue corresponding to position 8 thereof, and a nucleic acid encoding the peptide (b) that comprises an amino acid sequence having an alanine residue as an amino acid residue corresponding to position 2 of the amino acid sequence represented by SEQ ID NO: 1, a valine residue as an amino acid residue corresponding to position 3 thereof, an isoleucine residue as an amino acid residue corresponding to position 5 thereof, and/or a leucine residue as an amino acid residue corresponding to position 7 thereof. More specific examples thereof include a nucleic acid comprising a nucleotide sequence encoding the active domain of the BIL3 paralog or ortholog consisting of any of the amino acid sequences represented by SEQ ID NOs: 2 to 7. Alternatively, the nucleic acid may be a nucleic acid comprising a nucleotide sequence encoding the active domain consisting of an amino acid sequence represented by any of SEQ ID NOs: 30 to 33.

In the transgenic plant of the present embodiment, the active domain contained in the expressed active peptide functions as a peptide hormone and confers plant disease resistance and/or branching activity to the plant. For this purpose, at least the active domain contained in the expressed active peptide needs to be extracellularly transported. Hence, the active peptide preferably comprises an extracellular transport signal and a cleavage site for cleaving the signal peptide after extracellular transport. For example, the full-length BIL3 protein (FIG. 1) or the full-length protein of its paralog or ortholog comprises an extracellular transport signal and a cleavage site and is thus preferred as the active peptide that is expressed in the transgenic plant of the present embodiment. Thus, the nucleic acid according to the present embodiment is preferably BIL3 gene having the nucleotide sequence represented by SEQ ID NO: 19 or BIL3 paralog gene or ortholog gene having any of the nucleotide sequences represented by SEQ ID NOs: 20 to 29 and 38 to 41. In this context, the nucleotide sequences represented by SEQ ID NOs: 19 to 29 encode the amino acid sequences represented by SEQ ID NOs: 8 to 18, respectively. Alternatively, the nucleic acid is a nucleic acid that hybridizes under stringent conditions to the nucleic acid consisting of any of the nucleotide sequences described above and encodes the peptide having the activities described above. The stringent hybridization conditions are described in, for example, Sambrook, J. et al., (1989) Molecular Cloning: a Laboratory Manual Second Ed., Cold Spring Harbor Laboratory Press, Cold Spring Harbor, N.Y. Specific examples thereof include conditions involving incubation with a labeled probe at 68° C. in 6×SSC, 5×Denhardt's reagent, 0.5% SDS, and 100 μg/ml denatured and fragmented salmon sperm DNA, and washing starting at room temperature in 2×SSC and 0.1% SDS and continuing up to a salt concentration changed to 0.1×SSC and a temperature changed to 68° C. until background signals are no longer detected.

5-1-2. Nucleic Acid Expression System

The transgenic plant of the present embodiment comprises at least one exogenous nucleic acid expression system comprising, in an expressible state, a nucleic acid encoding the active peptide of the first embodiment.

The “exogenous nucleic acid expression system” refers to a foreign nucleic acid expression system transferred from outside via artificial operation. Thus, the exogenous nucleic acid expression system does not correspond to an endogenous nucleic acid expression system originally located at a predetermined locus on the plant genome. However, even such an endogenous nucleic acid expression system, when mutated from outside via artificial operation such as mutagenesis or whose origin is derived from an exogenous nucleic acid expression system, as in the progeny of the transgenic plant, is also included in the exogenous nucleic acid expression system of the present invention.

The “nucleic acid expression system” refers to one expression system unit that can cause expression of the nucleic acid (mainly, a gene or a fragment thereof) incorporated in the system. Thus, the nucleic acid expression system according to the present embodiment incorporates the nucleotide sequence of the nucleic acid encoding the active peptide serving as the active ingredient described in the first embodiment and can express the active peptide in the cell of the transgenic plant. The nucleic acid expression system has expression regulatory regions essential for gene expression, in addition to the nucleic acid region. The essential expression regulatory regions include, for example, a promoter and a terminator. The system may additionally comprise an enhancer, a poly A addition signal, a 5′-UTR (untranslated region) sequence, a marker or selective marker gene, a multicloning site, a replication origin, and the like. The nucleic acid expression system includes the whole of one expression system unit necessary for the expression of the particular gene or the like isolated from the genome as well as a system artificially constructed, for example, by combining expression regulatory regions or the like derived from various organisms. Any of these nucleic acid expression systems may be used in the present invention.

General plants also usually have endogenous BIL3 gene or its paralog gene or ortholog gene (hereinafter, referred to as a “BIL3 gene, etc.”). Hence, the plants have endogenous disease resistance and/or branching activity attributed to the expression of these genes. In order to allow the transgenic plant of the present embodiment to have more augmented disease resistance and/or branching activity compared with wild-type plants of the same species thereas, the nucleic acid expression system needs to express the BIL3 gene, etc. at a level exceeding the usual expression level. Thus, the nucleic acid expression system used in the present embodiment desirably has, for example, the property of being capable of overexpressing the incorporated nucleic acid encoding the active peptide and/or constitutively expressing or inducibly expressing the incorporated nucleic acid. This exogenous nucleic acid expression system may further have the property of being capable of maintaining a plurality of its own copies (multicopy) in the plant cell.

The nucleic acid expression system capable of overexpression can express the incorporated nucleic acid encoding the active peptide at 2 or more times, preferably 5 or more times, more preferably 10 or more times or 20 or more times, the expression level of the endogenous BIL3 gene, etc. per nucleic acid expression system.

The nucleic acid expression system capable of constitutive expression can continuously express the active peptide all the time, regardless of timing or an expression site. Hence, the nucleic acid expression system having this property is very effective because the system can provide the active peptide, etc. independently of the temporal or positional control, if any, of the expression of the endogenous BIL3 gene, etc.

In contrast to the nucleic acid expression system capable of constitutive expression, the nucleic acid expression system capable of inducible expression can express the active peptide in a time- or site-specific manner. Thus, this nucleic acid expression system is very effective when the endogenous BIL3 gene, etc. undergoes the temporal and/or site-specific control of expression or when the active peptide is expressed at the desired timing and the desired site.

The multicopy nucleic acid expression system has the advantage that, even if each individual nucleic acid expression system expresses the BIL3 gene, etc. at a low expression level, the nucleic acid expression system itself can increase in number, resulting in an increased expression level per cell as a whole. In the present embodiment, the multicopy nucleic acid expression system can be used in combination with the nucleic acid expression system capable of overexpression, the nucleic acid expression system capable of constitutive expression, or the nucleic acid expression system capable of inducible expression to thereby more effectively confer pathogen resistance and/or branching activity to the plant.

The constitution of the exogenous nucleic acid expression system having the property described above is not limited as long as the system has components essential for expression and incorporates the active peptide-encoding nucleic acid in an expressible state. In this context, the phrase “incorporating in an expressible state” means that the active peptide-encoding nucleic acid is expressibly inserted in the nucleic acid expression system. Specifically, this means that the active peptide-encoding nucleic acid is placed under the control of a promoter and a terminator in the nucleic acid expression system. Specific examples of the nucleic acid expression system thus constituted include expression vectors.

In the present invention, the “expression vector” refers to a nucleic acid expression system that can transport the incorporated active peptide-encoding nucleic acid, etc. into the plant cell of interest so that the active peptide can be expressed in the plant cell. Specific examples thereof include expression vectors based on plasmids or viruses.

In the case of the expression vector based on a plasmid (hereinafter, referred to as a “plasmid expression vector”), for example, a vector of pPZP, pSMA, pUC, pBR, pBluescript (Stratagene Corp.), or pTriEX™ (Takara Bio Inc.) series, or a binary vector of pBI, pRI, or pGW series can be used as a plasmid moiety.

In the case of the expression vector based on a virus (hereinafter, referred to as a “viral expression vector”), cauliflower mosaic virus (CaMV), bean golden mosaic virus (BGMV), tobacco mosaic virus (TMV), or the like can be used as a virus moiety.

The expression vector comprises, as described above, a promoter and a terminator as expression regulatory regions. The expression vector may additionally comprise an enhancer, a poly A addition signal, a 5′-UTR (untranslated region) sequence, a marker or selective marker gene, a multicloning site, a replication origin, and the like. The respective types of these components are not limited as long as these components can exert their functions in the plant cell. Those known in the art can be selected appropriately according to the plant to be transformed or according to purposes (e.g., expression pattern) in the plant.

The promoter used can be selected, for example, according to the desired expression pattern, from an overexpression-type promoter, a constitutive promoter, a site-specific promoter, a stage-specific promoter, and/or an inducible promoter. Specific examples of the overexpression-type constitutive promoter include cauliflower mosaic virus (CaMV)-derived 35S promoter, Ti plasmid-derived nopaline synthase gene promoter Pnos, maize-derived ubiquitin promoter, Oryza sativa-derived actin promoter, and tobacco-derived PR protein promoter. Ribulose bisphosphate carboxylase small subunit (Rubisco ssu) promoters or histone promoters of various plant species may be used. Specific examples of the site-specific promoter include root-specific promoter described in JP Patent Publication (Kokai) No. 2007-77677 A (2007).

Examples of the terminator include nopaline synthase (NOS) gene terminator, octopine synthase (OCS) gene terminator, CaMV 35S terminator, E. coli lipopolyprotein lpp 3′ terminator, trp operon terminator, amyB terminator, and ADH1 gene terminator. The terminator is not limited as long as its sequence can terminate the transcription of the gene transcribed by the action of the promoter.

Examples of the enhancer include an enhancer region containing an upstream sequence in the CaMV 35S promoter. The enhancer is not limited as long as the enhancer can enhance the expression efficiency of the active peptide-encoding nucleic acid, etc.

Examples of the marker or selective marker gene include drug resistance genes (e.g., tetracycline resistance gene, ampicillin resistance gene, kanamycin resistance gene, hygromycin resistance gene, spectinomycin resistance gene, chloramphenicol resistance gene, and neomycin resistance gene), fluorescent or luminescent reporter genes (e.g., luciferase, β-galactosidase, β-glucuronidase (GUS), and green fluorescence protein (GFP) genes), and enzyme genes such as neomycin phosphotransferase II (NPT II), dihydrofolate reductase, and Blasticidin S resistance genes. The marker or selective marker gene may be ligated with the same expression vector as the expression vector incorporating the active peptide-encoding nucleic acid, etc. or may be ligated with an expression vector different therefrom. In the latter case, the plant of interest can be co-transformed with both the expression vectors to thereby produce effects equivalent to those brought about by transformation with the single expression vector incorporating both the nucleic acid and the gene.

5-2. Method for Preparing Transgenic Plant

5-2-1. Preparation of Nucleic Acid Expression System

The nucleic acid expression system can be prepared according to a method known in the art, for example, a method described in Sambrook, J. et al., (1989) Molecular Cloning: a Laboratory Manual Second Ed., Cold Spring Harbor Laboratory Press, Cold Spring Harbor, N.Y.

Hereinafter, the preparation of the plasmid expression vector or the viral expression vector will be described with reference to specific examples, though the preparation of the nucleic acid expression system is not limited thereto.

(1) Preparation of Plasmid Expression Vector

First, of the nucleic acids described in the paragraph 5-1-1, the desired nucleic acid is cloned. For example, in the case of cloning the Arabidopsis thaliana BIL3 gene, an appropriate region is selected from the nucleotide sequence represented by SEQ ID NO: 19, and an oligonucleotide having the nucleotide sequence thereof is chemically synthesized. The chemical synthesis can utilize a custom synthesis service provided by each life science manufacturer.

Next, the BIL3 gene is isolated with the oligonucleotide as a probe from an Arabidopsis thaliana cDNA library according to a method known in the art, for example, plaque hybridization. For the details of the isolation method, see Sambrook, J. et al., (1989) (supra). Since the Arabidopsis thaliana cDNA library is commercially available from each life science manufacturer such as Stratagene Corp., such a commercially available product may be used. Alternatively, oligonucleotides serving as a primer pair may be chemically synthesized on the basis of the nucleotide sequence represented by SEQ ID NO: 19, and the BIL3 gene of interest can be amplified from an Arabidopsis thaliana genomic DNA or cDNA library by a nucleic acid amplification method such as PCR using the primer pair. In the case of performing the nucleic acid amplification, high-fidelity DNA polymerase having 3′-5′ exonuclease activity, such as Pfu polymerase, is preferably used. For detailed conditions, etc., for the nucleic acid amplification, see, for example, a method described in Innis M. et al (Ed.), (1990), Academic Press, PCR Protocols: A Guide to Methods and Applications. The isolated BIL3 gene is inserted, if necessary, to an appropriate plasmid and cloned in a host microbe such as E. coli. Then, its full-length nucleotide sequence is confirmed according to a technique known in the art.

Subsequently, the BIL3 gene is integrated into a predetermined site in the core of the desired nucleic acid expression system (backbone portion of the nucleic acid expression system). For example, the BIL3 gene is cleaved with appropriate restriction enzymes on the basis of the determined nucleotide sequence. Meanwhile, the nucleic acid expression system is cleaved at the corresponding restriction enzyme sites. A multicloning site, if present, in the nucleic acid expression system is conveniently used. Subsequently, the BIL3 gene is inserted to the nucleic acid expression system by the ligation of both the nucleic acids at their ends using ligase or the like to complete the nucleic acid expression system for the BIL3 gene of interest. These series of gene manipulation procedures are technically well known in the art. For the details of the method, see, for example, Sambrook, J. et al., (1989) (supra).

(2) Preparation of Viral Expression Vector

Basic procedures therefor can follow the method for preparing the plasmid expression vector. First, a plant virus genome is prepared by a method known in the art and then inserted to an appropriate cloning vector, for example, of E. coli-derived pBI, pPZP, pSMA, pUC, pBR, or pBluescript series, to obtain a recombinant. Next, the active peptide-encoding nucleic acid is inserted to a predetermined site in the viral genome contained in the recombinant, and cloned. Subsequently, a plant virus genome region can be excised from the recombinant using restriction enzymes. In this way, the viral expression vector of interest can be obtained.

(3) Method for Transferring Nucleic Acid Expression System into Plant Cell

The nucleic acid expression system incorporating the active peptide-encoding nucleic acid can be transferred into a plant cell, i.e., a plant cell can be transformed, by an arbitrary appropriate method known in the art. In the case of using a plasmid expression vector as the nucleic acid expression system, for example, a protoplast, particle gun, or Agrobacterium method can be used preferably as the transformation method.

The protoplast method involves removing cell walls from plant cells by enzymatic (e.g., cellulase) treatment and transferring the gene of interest into the resulting plant cells (protoplasts). This method can be further classified into electroporation, microinjection, and polyethylene glycol methods, etc., depending on an approach for the gene transfer. The electroporation method involves applying an electric pulse to a mixed solution of the protoplast and the gene of interest to transfer the gene into the protoplast. The microinjection method involves directly transferring the gene of interest into the protoplast under a microscope using a microneedle. The polyethylene glycol method involves transferring the gene of interest to the protoplast by the action of polyethylene glycol.

The particle gun method involves attaching the gene of interest to microparticles of gold, tungsten, or the like, and intracellularly injecting the resulting microparticles into plant tissues using high-pressure gas to transfer the gene of interest into the cells. This method can produce transformed cells containing the gene of interest integrated in the genomic DNA of the recipient plant cells. The successfully transformed cells are usually selected on the basis of the marker gene product in the nucleic acid expression system.

The Agrobacterium method involves transforming plant cells using, as transforming factors, a bacterium of the genus Agrobacterium (e.g., A. tumefaciens and A. rhizogenes) and Ti plasmid derived therefrom. This method can transfer the gene of interest into the genomic DNA of recipient plant cells.

These methods are all known in the art. For the details thereof, see appropriate protocols described in, for example, Shokubutsu Taisha Kogaku (Plant Metabolic Engineering in English) Handbook (2002, NTS Inc.) or Shinban Model Shokubutsu No Jikken Protocol: Idengakuteki Shuhou Kara Genome Kaiseki Made (Experimental Protocol of Model Plant, New Edition: From Genetic Technique To Genomic Analysis in English) (2001, Shujunsha Co., Ltd.).

Alternatively, in the case of using a viral expression vector (e.g., using CaMV, BGMV, or TMV described above) as the nucleic acid expression system, the plant cells of interest can be infected with the viral expression vector containing the integrated active peptide-encoding nucleic acid to obtain transformed cells. For the details of such a gene transfer method using the viral vector, see, for example, the method of Hohn et al. (Molecular Biology of Plant Tumors (Academic Press, New York) 1982, pp. 549) and U.S. Pat. No. 4,407,956.

The present invention does not necessarily require that the plant species from which the active peptide-encoding nucleic acid is derived should be identical to the plant species of plant cells to be transformed. For example, the cells of tobacco (Nicotiana tabacum) of the family Solanaceae may be transformed with the nucleic acid expression system incorporating the BIL3 gene derived from Arabidopsis thaliana of the family Brassicaceae. This is because: the BR signaling pathway involving BIL3 universally exists in plants; and, as shown in FIG. 2, each individual BIL3 peptide hormone (active domain) is highly conserved across species; thus, even if a plant species is transformed with a nucleic acid expression system comprising a nucleic acid encoding an active peptide derived from species different therefrom, the nucleic acid expression system transferred to the plant species can exert functions similar to those brought about by the transformation of the plant species with a system derived from the same species thereas.

In the present invention, preferably, the plant to be transformed with the nucleic acid expression system has at least a wild-type gene cluster involved in the BR signaling pathway and a wild-type gene cluster involved in the natural immunity-inducing signaling pathway mediated by salicylic acid. This is because: the augmentation of plant disease resistance of the present invention is based on the augmented signals of these pathways; and even if signals are augmented by increasing the expression level of a polypeptide located upstream in the signaling pathway, subsequent signals cannot be transduced in the presence of its downstream factor having a loss-of-function mutation or the like, resulting in unsuccessful obtainment of the disease-resistant plant.

In the present invention, two or more nucleic acid expression systems differing in the active peptide-encoding nucleic acid incorporated therein can be transferred, in a range that can coexist with each other, into one plant cell. Two nucleic acid expression systems, for example, a nucleic acid expression system incorporating the Oryza sativa BIL3 ortholog gene and a nucleic acid expression system incorporating the Arabidopsis thaliana BIL3 gene, may be transferred into one plant cell (e.g., Oryza sativa cell).

After this step, the transformed plant cell can be regenerated into a transgenic plant according to a method known in the art. Examples thereof include an in vitro regeneration method which involves regenerating plant bodies through the formation of callus composed of undifferentiated grown cells. This method is known in the art. For the details thereof, see, for example, Shokubutsu Taisha Kogaku (Plant Metabolic Engineering in English) Handbook (2002, NTS Inc.) or Shinban Model Shokubutsu No Jikken Protocol: Idengakuteki Shuhou Kara Genome Kaiseki Made (Experimental Protocol of Model Plant, New Edition: From Genetic Technique To Genomic Analysis in English) (2001, Shujunsha Co., Ltd.) described above. Alternatively, an in planta method may be used, which involves directly transferring the nucleic acid expression system to the cells of the plant individual of interest without the callus or cell culture step. A phytohormone such as auxin, gibberellin, and/or cytokinin may be used for promoting the growth and/or division of the transformed cells.

The first-generation transgenic plant thus obtained by the method is a disease-resistant and/or branching-active plant. In the present invention, this first-generation transgenic plant also encompasses clones having genetic information identical thereto. The clones correspond to, for example, a plant obtained by the cutting, grafting, or layering of a portion of the plant body collected from the first-generation transgenic plant, a plant body regenerated through callus formation from cultured cells, or a vegetative plant newly formed vegetative reproductive organs (e.g., rhizomes, tuberous roots, corms, and runners) obtained by asexual reproduction from the first-generation transgenic plant.

The active peptide is expressed from the nucleic acid expression system transferred to this transgenic plant. The expression level of the active peptide per cell of the plant is increased compared with the wild-type individual of the same species thereas. As a result, the natural immune system is enhanced to improve disease resistance, while branching activity is augmented to increase the number of branching.

6. Progeny of Transgenic Plant

The sixth embodiment of the present invention relates to progeny of the transgenic plant. In the present specification, the “progeny of the transgenic plant” refers to offspring that is obtained via sexual reproduction from the first-generation transgenic plant of the fifth embodiment and intracellularly retains the nucleic acid expression system described in the fifth embodiment. The progeny corresponds to, for example, a seedling of the first-generation transgenic plant.

The progeny can be obtained from the transgenic plant of the fifth embodiment by a method known in the art. For example, the first-generation transgenic plant is allowed to fruit, and seeds can be obtained as first-generation progeny and also as a second-generation transgenic plant. As an example of a method for further obtaining second-generation progeny from the first-generation progeny of the present invention, the seeds are rooted on an appropriate medium, and the resulting shoots are transplanted in a pot containing soil. The second-generation progeny can be obtained by growth under appropriate cultivation conditions. The progeny of the present embodiment is not limited by its generation as long as the progeny retains the nucleic acid expression system described in the second embodiment. Thus, third or later generation progeny can be obtained by repeating a method similar to the method for obtaining the second-generation progeny.

### EXAMPLES

### Example 1

### Phenotyping of Bil3 Mutant

A semidominant bil3 mutant selected from activation tagging lines (Nakazawa M. et al., (2003) Plant J., 34: 741-750) because of its phenotype as Brz resistance and hypocotyl elongation in the dark place was observed and functionally analyzed.

(Method)

A plurality of seeds of the wild-type strain or bil3 mutant of Arabidopsis thaliana were inoculated over a ½ MS agar medium (½× Murashige & Skoog Medium Including Vitamins (Duchefa Biochemie B.V.)/1.5% Sucrose, pH 5.6), then placed in a dark box, and left standing at 4° C. for 2 days or longer. Then, the seeds were continuously irradiated with 100 μmoL/m²s white light at 22° C. for 4 hours and grown in the dark place again at 22° C. for 7 days. After light irradiation again for 2 days in the bright place, the seedlings were transplanted to soil. Six seedlings per pot were transplanted so that their hypocotyls and roots were completely buried under the ground. The soil used was 1 bag of horticultural soil supplemented with approximately 2 L of vermiculite and then sterilized by autoclaving. The seedlings were grown at 22° C. under long-day conditions (16-hour bright pace/8-hour dark place). Four weeks after the transplantation to soil, the morphology of plant bodies and the numbers of flower stalks and branches were measured for 10 individuals each of the wild-type strain and the bil3 mutant.

(Results)

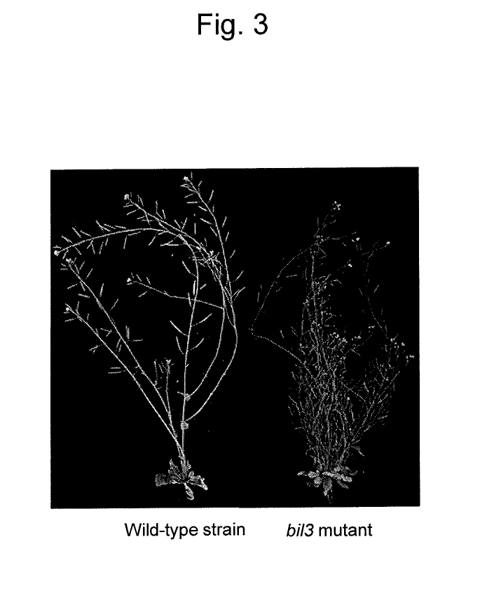

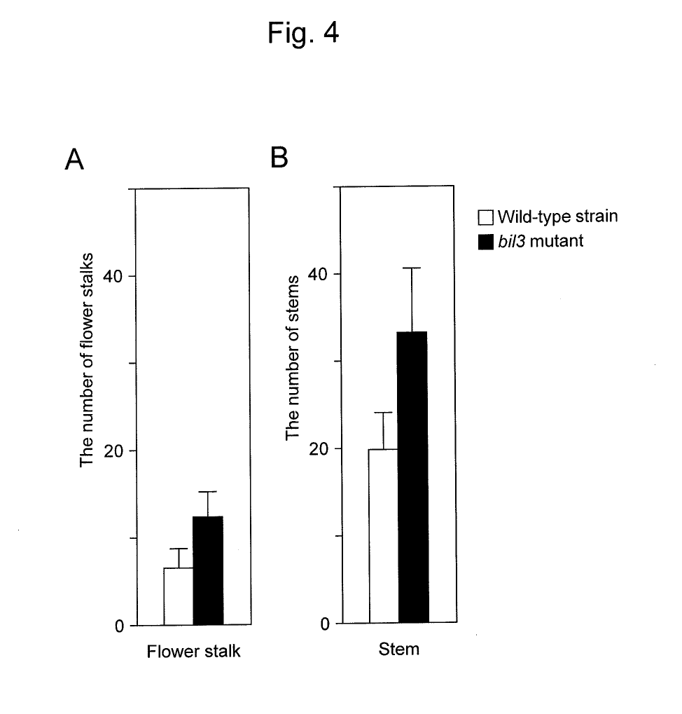

FIG. 3 shows the morphology of the aerial parts of the wild-type strain and the bil3 mutant. FIG. 4 shows the numbers of flower stalks (FIG. 4A) and branches (FIG. 4B) in the wild-type strain and the bil3 mutant. The bil3 mutant exhibited significant increases in the number of flower stalks by approximately 2 times and in the number of branches by approximately 1.6 times, compared with the wild-type strain.

### Example 2

### Expression Level Analysis of BIL3 Gene in Bil3 Mutant

Since the bil3 mutant is a semidominant mutant, the phenotypes of the bil3 mutant confirmed in Example 1 were presumably due to the overexpression of the BIL3 gene resulting from tag insertion. Thus, the expression level of the BIL3 gene in the bil3 mutant was analyzed by real-time PCR.

(Method)

Total RNA was extracted from each of the bil3 mutant and the wild-type strain using RNeasy Plant Mini Kit (Qiagen N.V.). First, less than 0.1 mg (fresh weight) of rosette leaves was collected from each plant, then frozen in liquid nitrogen, and then disrupted using a mortar. To the disrupted sample, 450 μL, of a β-mercaptoethanol (10 μL/buffer RLT (1 μL) mixed solution was added, and the mixture was vortexed. Specific procedures therefor followed the protocol included in the kit. Finally, total RNA obtained by ethanol precipitation was dissolved in 50 μL of RNase-free water.

Subsequently, cDNA was synthesized from the extracted total RNA using TaKaRa PrimeScript RT reagent Kit (Perfect Real Time). Specific procedures therefor followed the protocol included in the kit. The synthesized cDNA was diluted 10-fold, and the diluted solution was used as cDNA for real-time PCR. The primers used were At1g49500-RT-F (SEQ ID NO: 42) and At1g49500-RT-R (SEQ ID NO: 43) specifically amplifying the BIL3 gene. PCR reaction conditions involved preparing 30 μL in total of a reaction solution (12.5 μL at of SYBR Premix Ex Taq™ II; 0.1 μL each of 100 μM At1g49500-RT-F and -R primers; 5 μL of cDNA; and water), and treating the reaction solution at 95° C. for 30 seconds, followed by 40 cycles each involving 95° C. for 5 seconds and 60° C. for 30 seconds. A calibration curve was prepared using the dilution series of cDNA for real-time PCR.

(Results)

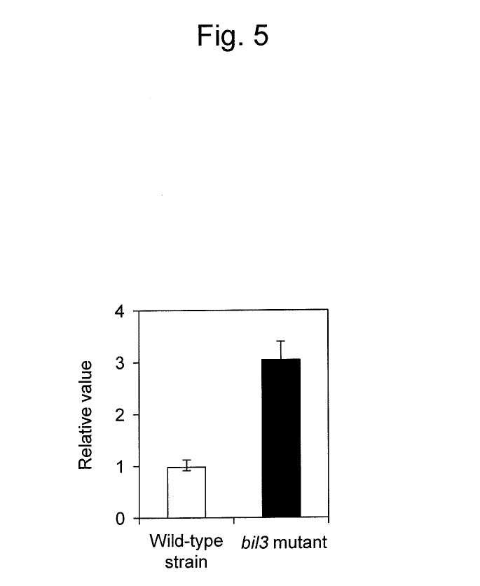

The results are shown in FIG. 5. As shown in this diagram, the bil3 mutant was shown to overexpress the BIL3 gene compared with the wild-type strain. This suggested that the phenotypes of the bil3 mutant confirmed in Example 1 were induced by the overexpression of the BIL3 gene.

### Example 3

### Preparation of BIL3-Overexpressing Transgenic Plant and Phenotyping Thereof

The results of Example 2 suggested that the phenotypes of the bil3 mutant were induced by the overexpression of the BIL3 gene. Thus, in order to verify this, transgenic Arabidopsis thaliana overexpressing the BIL3 gene linked downstream of 35S CaMV promoter was prepared and phenotyped.

(Method)

(1) Cloning of Wild-Type BIL3 Gene

First, total RNA extraction and cDNA synthesis for cDNA library preparation were performed according to the method of Example 2.

Next, in order to obtain the full-length ORF of the BIL3 gene, PCR reaction was performed using the prepared cDNA library and KOD-plus-DNA polymerase (Toyobo Co., Ltd.). The BIL3 primers used were primers bil3-GW-F (SEQ ID NO: 44) and bil3-GW-R (SEQ ID NO: 45) designed for the 5′ terminus and 3′ terminus of the BIL3 gene, respectively. The BIL3 gene was cloned using pENTR/D TOPO cloning kit (Invitrogen Corp.). Specific procedures therefor followed the protocol included in the kit. The nucleotide sequence of the subcloned BIL3 gene was confirmed by cycle sequencing using BigDye Terminator Cycle Sequencing Kit (Applied Biosystems, Inc.). In this way, a BIL3 gene entry clone pENTR-BIL3 was obtained.

Recombination was performed between the entry clone and a destination vector pGWB2 (Nakagawa et al., 2007, JBB, 104: 34-41) through LR reaction according to the Gateway technique to prepare an expression vector pGWB5-BIL3 for plant transformation (Gateway Vector) containing an insert of the gene of interest in the destination vector. A mixed solution of pENTR-BIL3, the pGWB2 vector, 5×LR Reaction Buffer, topoisomerase I, and LR Clonase™ was prepared and left standing at 25° C. for 1 hour. Then, 1 μL of protease K was added thereto, and the mixture was left standing at 37° C. for 10 minutes. Subsequently, the reaction solution was mixed with DH5α competent cells, and the mixture was left standing for 30 minutes on ice. Then, heat shock was applied at 2° C. for 30 seconds to the cells, which were immediately transferred onto ice and left standing for 2 minutes. The cells were applied to an LB medium containing 50 μL/mL each of kanamycin and hygromycin and cultured overnight. Plasmids were extracted from the resulting transformants to obtain the pGWB5-BIL3 vector of interest.

(2) Preparation of Transgenic Arabidopsis Thaliana

1 μL of pGWB2-BIL3 with respect to 200 μL of Agrobacterium competent cells (C58) was added thereto, then well mixed, and left standing for 30 minutes in ice. Subsequently, the cells were left standing for 1 minute in liquid nitrogen and then thawed in a block incubator set to 37° C. After addition of 1 mL of a YEP medium, the cells were cultured at 28° C. for 2 to 4 hours with shaking at 200 rpm. The cells were spread over a YEP medium containing 50 μg/mL each of kanamycin and hygromycin and 100 μg/mL rifampicin and cultured at 28° C. for 2 to 3 days. The successful transformation with the vectors was confirmed by colony PCR.

The colony thus transformed with the vector was precultured overnight at 28° C. in a YEP liquid medium. The volume of the culture solution was brought up to 500 ml by the addition of a YEP medium, followed by overnight culture. The culture solution was centrifuged at 5000 rpm for 10 minutes, and the supernatant was discarded. After addition of 400 mL of an infiltration medium (MS medium, 1000× Gamborg's vitamin, sucrose, benzylaminopurine, silwet, pH 5.7), the pellet was suspended at 28° C. at 177 rpm/minute for approximately 20 minutes. The suspension was transferred to a 300-mL beaker. A pot where 6 individuals of the wild-type strain grew was placed upside-down so that the plant bodies were dipped in the infiltration medium. In this state, the plants were left standing for 20 minutes. The plant bodies were wrapped in plastic wrap and left overnight. The plant bodies were grown, and the obtained seeds were inoculated to an MS medium containing 25 μg/mL kanamycin to select T1 transformants. The obtained T1 seeds were inoculated to an MS medium containing 20 μg/mL kanamycin to select T2 transformants. The obtained transformant was designated as a “35S::BIL3 transformant” and grown in the same way as in Example 1, followed by morphological observation thereof.

The expression of the BIL3 gene was further analyzed by real-time PCR. The real-time PCR was performed according to the method of Example 2.

(Results)

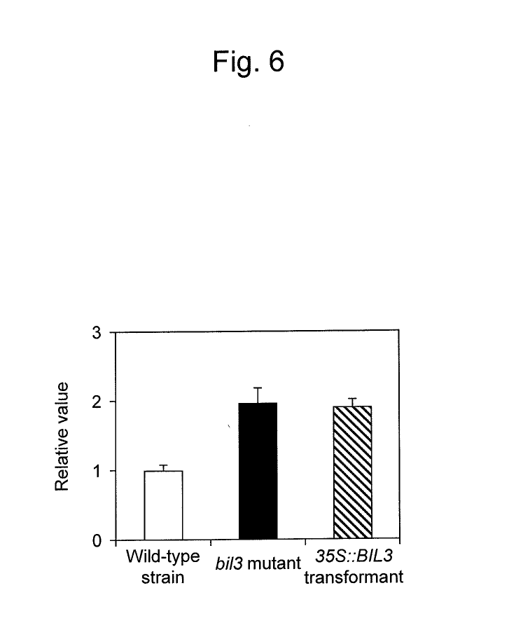

Fifty 35S::BIL3 transformants were obtained as BIL3-overexpressing transgenic strains. As shown in FIG. 6, the 35S::BIL3 transformants were confirmed to express the BIL3 gene at a level equivalent to that of the bil3 mutant and have a significantly higher expression level thereof than that of the wild-type strain. Also, increases in the number of flower stalks and the number of branches similar to those of the bil3 mutant were observed in all the 35S::BIL3 transformants.

These results demonstrated that increases in the number of plant flower stalks and the number of branches, i.e., the number of branching, observed in the bil3 mutant were due to the overexpression of the BIL3 gene. This showed that BIL3 is a protein having the activity of augmenting plant branching.

### Example 4

### Induction of PR1 Gene Expression in Bil3 Mutant

The gene expression of a pathogen resistance marker PR1 (pathogenesis related 1D) was tested in the bil3 mutant. PR1 is an antibacterial protein whose expression is induced by the bacterial pathogen infection of a plant, and is known to typically function downstream in the pathogen resistance signaling pathway mediated by salicylic acid. Hence, the increased expression of the marker PR1 gene in a plant can serve as an index showing that the plant has disease resistance even without bacterial pathogen infection. Meanwhile, Nakashita et al. (The Plant Jour., 2003, 33: 887-898) have found that BR increases the expression of the PR1 gene, showing the presence of a BDR-inducing pathway that is different from the induced disease resistance mediated by salicylic acid. Thus, the increased expression of the PR1 gene in the bacterial pathogen-uninfected bil3 mutant suggests that this bil3 strain has acquired disease resistance.

(Method)

Individuals of the wild-type strain and the bil3 mutant grown in the same way as in Example 1 were treated or untreated with brassinolide (BL), one type of BR, to prepare samples. Total RNA was extracted from the rosette leaf of each strain using RNeasy Plant Mini Kit (Qiagen N.V.). Next, cDNA was synthesized from the extracted total RNA using PrimeScript First-Strand cDNA Synthesis Kit (Takara Bio Inc.). Specific procedures for the total RNA extraction and the cDNA synthesis followed the protocol included in each kit. Subsequently, the expression of the PR1 gene was analyzed by real-time PCR with the synthesized cDNA as a template using primers PR1-RT-F represented by SEQ ID NO: 46 and PR1-RT-R represented by SEQ ID NO: 47, SYBR Premix EX Taq kit (Takara Bio Inc.) and a real-time PCR apparatus Thermal Cycler Dice (Takara Bio Inc.).

(Results)

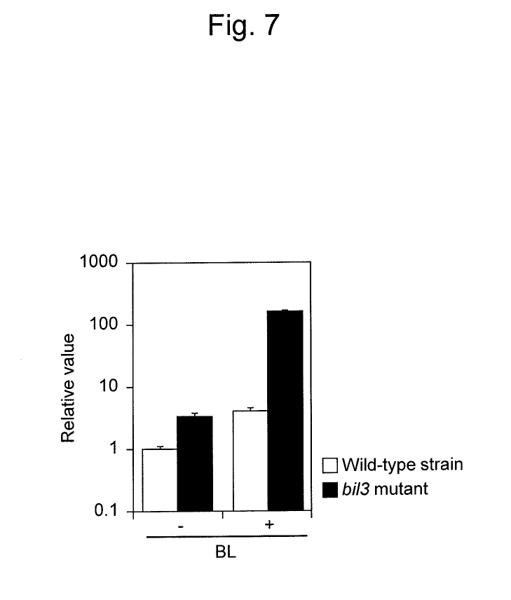

The results are shown in FIG. 7. In this diagram, the expression level of the PR1 gene is indicated by relative value with the value of the BL-untreated (BL-) wild-type strain as 1. The expression level of PR1 was increased by approximately 3 times even in the BL-untreated bil3 mutant and by approximately 40 times in the BL-treated bil3 mutant, compared with the wild-type strain. This suggests that the bil3 mutant acquired disease resistance. As shown in Example 2, the bil3 mutant is a BIL3 gene-overexpressing mutant. Thus, this result shows that BIL3 is also involved in the BR intracellular signaling pathway and the induction of PR1 gene expression, i.e., the induction of disease resistance, in the bil3 mutant is attributed to BIL3.

All publications, patents, and patent applications cited herein are incorporated herein by reference in their entirety.

1. A peptide or a salt thereof having activities of at least one of augmenting plant disease resistance or branching consisting of
(a) the amino acid sequence represented by SEQ ID NO: 1, or
(b) an amino acid sequence having identity to 4 or more amino acids in the amino acid sequence of SEQ ID NO: 1 and having an alanine residue or a serine residue as an amino acid residue corresponding to position 2 of the amino acid sequence of SEQ ID NO: 1,
nonpolar amino acid residues as amino acid residues corresponding to positions 3, 5, 7, and 8 of the amino acid sequence of SEQ ID NO: 1,
proline residues as amino acid residues corresponding to positions 4 and 6 of the amino acid sequence of SEQ ID NO: 1, and
a glycine residue as an amino acid residue corresponding to position 9 of the amino acid sequence of SEQ ID NO: 1.

2. The peptide or salt thereof of claim 1, wherein the amino acid sequence (b) further comprises at least one of
a valine residue, an isoleucine residue, or a proline residue as an amino acid residue corresponding to position 3 of the amino acid sequence of SEQ ID NO: 1,
an isoleucine residue or a valine residue as an amino acid residue corresponding to position 5-of the amino acid sequence of SEQ ID NO: 1,
a leucine residue or a phenylalanine residue as an amino acid residue corresponding to position 7-of the amino acid sequence of SEQ ID NO: 1, or
a valine residue as an amino acid residue corresponding to position 8-of the amino acid sequence of SEQ ID NO: 1.

3. The peptide or salt thereof of claim 1, wherein the amino acid sequence (b) comprises at least one of
an alanine residue as an amino acid residue corresponding to position 2 of the amino acid sequence of SEQ ID NO: 1,
a valine residue as an amino acid residue corresponding to position 3 of the amino acid sequence of SEQ ID NO: 1,
an isoleucine residue as an amino acid residue corresponding to position 5 of the amino acid sequence of SEQ ID NO: 1, or
a leucine residue as an amino acid residue corresponding to position 7-of the amino acid sequence of SEQ ID NO: 1.

4. The peptide or salt thereof of claim 1, wherein the peptide having the amino acid sequence (b) consists of any of the amino acid sequences of SEQ ID NOs: 2 to 7.

5. The peptide or salt thereof of claim 1, wherein the peptide consists of any of the amino acid sequences of SEQ ID NOs: 8 to 18.

6. A composition for conferring at least one of disease resistance to a plant or for augmenting plant branching, comprising at least one of the peptide of claim 1, the peptide of claim 1 further having at least one amino acid added to the N terminus or the C terminus thereof and salts thereof as an active ingredient.

7. A method for suppressing microbial infection in a plant comprising the step of allowing the peptide or the salt thereof of claim 1 to act on the plant.

8. A method for augmenting plant branching comprising the step of allowing the peptide or the salt thereof of claim 1 to act on the plant.

9. A plant with conferred disease resistance or augmented branching comprising at least one exogenous nucleic acid expression system comprising in an expressible state, a nucleic acid encoding a peptide having activities of at least one of augmenting plant disease resistance or branching and:
(a) comprising the amino acid sequence represented by SEQ ID NO: 1; or
(b) comprising an amino acid sequence having identity to 4 or more amino acids in the amino acid sequence of SEQ ID NO: 1 and having
an alanine residue or a serine residue as an amino acid residue corresponding to position 2 of the amino acid sequence represented by SEQ ID NO: 1,
nonpolar amino acid residues as amino acid residues corresponding to positions 3, 5, 7, and 8 of the amino acid sequence of SEQ ID NO: 1,
proline residues as amino acid residues corresponding to positions 4 and 6 of the amino acid sequence of SEQ ID NO: 1, and
a glycine residue as an amino acid residue corresponding to position 9 of the amino acid sequence of SEQ ID NO: 1.

10. The plant of claim 9, wherein the amino acid sequence of the peptide (b) further comprises
a valine residue, an isoleucine residue, or a proline residue as an amino acid residue corresponding to position 3 of the amino acid sequence of SEQ ID NO: 1,
an isoleucine residue or a valine residue as an amino acid residue corresponding to position 5-of the amino acid sequence of SEQ ID NO: 1,
a leucine residue or a phenylalanine residue as an amino acid residue corresponding to position 7-of the amino acid sequence of SEQ ID NO: 1, or
a valine residue as an amino acid residue corresponding to position 8 of the amino acid sequence of SEQ ID NO: 1.

11. The plant of claim 9, wherein the amino acid sequence of the peptide (b) comprises
an alanine residue as an amino acid residue corresponding to position 2 of the amino acid sequence of SEQ ID NO: 1,
a valine residue as an amino acid residue corresponding to position 3 thereof,
an isoleucine residue as an amino acid residue corresponding to position 5 thereof, or
a leucine residue as an amino acid residue corresponding to position 7 thereof.

12. The plant of claim 9, wherein the peptide (b) consists of any of the amino acid sequences of SEQ ID NOs: 2 to 7.

13. The plant of claim 9, wherein the peptide (b) consists of any of the amino acid sequences of SEQ ID NOs: 8 to 18.

14. The plant of claim 9, wherein the nucleic acid expression system overexpresses the nucleic acid incorporated therein.

15. The plant of claim 9, wherein the nucleic acid expression system constitutively expresses the nucleic acid incorporated therein.

16. The plant of claim 9, wherein the nucleic acid expression system inducibly expresses the nucleic acid incorporated therein.

17. The plant of claim 9, wherein the nucleic acid expression system is an expression vector.

18. The progeny of a plant of claim 9.

19. A method for suppressing microbial infection in a plant comprising the step of allowing the composition of claim 6 to act on the plant.

20. A method for augmenting plant branching comprising the step of allowing the composition of claim 6 to act on the plant.
